(19) United States
(12) Patent Application Publication
(10) Pub. No.: US 2021/0267959 A1
(43) Pub. Date: Sep. 2, 2021

(54) METHODS OF TREATING RENAL DISEASE

(71) Applicants: The Regents of the University of California, Oakland, CA (US); The United States Government as Represented by the Department of Veterans Affairs, Washington, DC (US)

(72) Inventors: Hamid MORADI, Laguna Hills, CA (US); Daniele PIOMELLI, Irvine, CA (US); Kamyar KALANTAR-ZADEH, Palos Verdes Estates, CA (US)

(21) Appl. No.: 17/260,188

(22) PCT Filed: Jul. 16, 2019
(86) PCT No.: PCT/US2019/042029

§ 371 Date: Jan. 13, 2021

Related U.S. Application Data
(60) Provisional application No. 62/699,442, filed on Jul. 17, 2018.

(51) Int. Cl. A61K 31/4525; A61P 13/12

(52) U.S. Cl. CPC A61K 31/4525; A61P 13/12

(57) ABSTRACT

Disclosed herein, inter alia, are methods of treating renal disease (e.g., chronic kidney disease or end stage renal disease).

### CROSS-REFERENCES TO RELATED APPLICATIONS

This application claims the benefit of U.S. Provisional Application No. 62/699,442, filed Jul. 17, 2018, which is incorporated herein by reference in its entirety and for all purposes.

### BACKGROUND OF THE INVENTION

While mortality in patients with end-stage renal disease (ESRD) is exceptionally high, traditional risk factors such as obesity are paradoxically associated with better survival whereas nontraditional risk factors including cachexia increase the likelihood of poor outcomes.

The prevalence of chronic kidney disease (CKD) in the United States (U.S.) continues to rise with recent projections estimating that approximately 25 million patients have moderate to severe CKD (stage III-V) and more than 450,000 have ESRD requiring renal replacement therapy. (1) Furthermore, there is evidence that the occurrence of this disorder is on the rise worldwide. (2,3) It is well known that patients with CKD have a significantly increased risk of all-cause and cardiovascular mortality, especially in those with ESRD on renal replacement therapy. In spite of many recent improvements in dialysis treatment and the adherence of patients and physicians to the quality measures set forth by guidelines, ESRD patients on maintenance hemodialysis (MHD) continue to experience an annual mortality rate of approximately 20%, a rate worse than many cancers. (1) The risk factors responsible for this disproportionately elevated risk of death in MHD patients have not been fully identified. In fact, traditional risk factors such as obesity and hypertriglyceridemia cannot explain the magnitude of the risk observed in these patients given that they are paradoxically associated with better survival in observational studies of hemodialysis patients. (4,5) In addition, there is accumulating evidence that nontraditional risk factors, such as cachexia and impaired energy metabolism, may play a more prominent role in the higher risk of mortality in patients with ESRD. (6,7)

ESRD is associated with a catabolic state marked by increased basal energy expenditure which leads to wasting of adipose tissue and skeletal muscle. (6,7) The nutritional and metabolic derangements in patients with ESRD that lead to cachexia and wasting are collectively described as protein energy wasting (PEW). (6) There are reports indicating that up to 75% of patients with ESRD show signs of wasting and cachexia. (8) In addition, the presence of cachexia is associated with poor outcomes including a significantly higher risk of death. (6) Numerous pathways have been implicated in the pathogenesis of cachexia and PEW in ESRD. These include inflammation, oxidative stress, uremia, anorexia, dialysis-related catabolic state and more recently browning of white adipose tissue. (6,9) Rats with CKD show an increased expression of genes involved in energy expenditure rather than storage as seen in brown adipose tissue. This was associated with muscle and fat wasting and cachexia through inefficient energy expenditure. (10) While there are many reports on potential causes of cachexia in ESRD, there is a paucity of data on factors/pathways that might play a compensatory role and counteract the effects of wasting in this patient population.

One promising area that has not been fully explored is the role of the endocannabinoid (EC) system in cachexia and mortality of ESRD. This system is composed of endogenous, bioactive lipid-derived mediators, the endocannabinoids, which exert their effects through specific G protein-coupled receptors: cannabinoid-1 (CB₁) and cannabinoid-2 (CB₂). The most extensively studied ECs are anandamide (AEA) and 2-arachidonoyl-sn-glycerol (2-AG). (11,12) The EC system plays important roles in many different physiologic processes, and CB₁ and CB₂ receptors have been discovered in a multitude of peripheral organ systems, including white adipose tissue. (13) In particular, this system contributes in important ways to energy metabolism by overseeing energy requirements and expenditure via a multitude of central and peripheral mechanisms. (11,14) For instance, activation of the EC system leads to increased intake of energy-rich foods, decreased energy expenditure via promoting white adipogenesis and inhibition of brown adipose tissue activation. (15) In addition, activation of this system stimulates molecular pathways involved in energy storage including fatty acid production and lipogenesis. Therefore, it is not surprising that overactivity of the EC system can lead to obesity, hypertriglyceridemia and metabolic syndrome in animals and humans. (11,14,15) Indeed, many recent studies in patients with obesity and metabolic syndrome have found significant elevations of serum ECs, and there has been extensive work demonstrating a causative relationship between abnormal EC system activity and development of metabolic syndrome. (16,18) Conversely, pharmacological antagonists of CB₁ receptors have been shown to decrease body weight and improve metabolic profile in obese animals and humans. (19,20,21) However, the impact of cachexia and wasting on the EC system, and vice versa, remains to be fully elucidated. Disclosed herein, inter alia, are solutions to these and other problems in the art.

### BRIEF SUMMARY OF THE INVENTION

In an aspect is provided a method of treating chronic kidney disease in a subject in need thereof, the method including administering an effective amount of an agent that increases the level of activity of a cannabinoid receptor, to the subject.

In an aspect is provided a method of treating chronic kidney disease in a subject in need thereof, the method including administering an effective amount of an agent that increases the serum level of 2-arachidonoyl-sn-glycerol (2-AG), to the subject.

In an aspect is provided a method of identifying a subject for treatment with a method described herein, including detecting the serum level of 2-arachidonoyl-sn-glycerol (2-AG) in a candidate subject.

### BRIEF DESCRIPTION OF THE DRAWINGS

FIG. 1A-1B. Comparison of Serum AEA and 2-AG Concentrations in MHD Patients and Control Subjects. (FIG. 1A) Serum AEA in 50 MHD Patients and 21 Control Subjects. (FIG. 1B) Serum 2-AG in 50 MEM Patients and 21 Control Subjects. Serum 2-AG levels are presented on a logarithmic scale for visual purposes only.

FIG. 2 Association of Serum 2-AG and All-Cause Mortality in 96 MHD Patients. Serum 2-AG levels are presented on a logarithmic scale for visual purposes only. Model 1: Unadjusted; Model 2: Adjusted for case-mix variables, which included age, gender, race, and ethnicity; Model 3: Adjusted for covariates in Model 2, plus diabetes and dialysis vintage; Model 4: Adjusted for covariates in Model 3, plus inflammation (serum IL-6).

FIG. 3. Potential Impact of Increased Serum 2-AG Levels in Patients with ESRD on MHD.

FIG. 4. Concentration of serum AG in 21 controls, 50 MHD, 13 PD and 6 CKD patients. Serum AG levels are presented on a logarithmic scale for visual purposes only.

FIG. 5 Concentration of Serum AG in 96 MHD patients and 21 controls. Serum AG levels are presented on a logarithmic scale for visual purposes only.

FIG. 6. Cohort construction

FIG. 7. Distribution of Serum AG in 96 MHD patients. Distribution of serum AG level in 96 HD patients at the time of measurement.

FIG. 8. Administration of intraperitoneal JZL184 in a rat and mouse model of chronic kidney disease (CKD).

FIG. 9. Treatment with JZL184 and effect on renal and cerebral cortex 2-AG concentration in rats.

FIG. 10. Treatment with JZL184 and effect on blood pressure, serum BUN concentration and urinary protein excretion in rats. *p<0.05, **p<0.01, *** p<0.001, ****p<0.0001

FIG. 11. Male C57BL/6J mice underwent sham surgery to induce CKD then were treated with vehicle or JZL184 therapy (4 mg/kg).

FIG. 12. Treatment with JZL184 and effect on blood pressure, serum BUN concentration and urinary protein excretion in mice. *p<0.05, **p<0.01, *** p<0.001, ****p<0.0001.

FIG. 13. Increasing tissue 2-AG levels and rate of metabolism (n=5 in each group).

FIGS. 14A-14B. Increasing serum 2-AG levels are associated with reduced risk of death in patients on maintenance hemodialysis. Restricted cubic splines of the association between serum 2-AG and 12-month all-cause mortality among 400 maintenance hemodialysis patients. Splines were adjusted for covariates: FIG. 14A: age, gender, race and ethnicity, diabetes and dialysis vintage. FIG. 14B age, gender, race and ethnicity, diabetes, dialysis vintage and serum IL-6 levels. Solid and dotted lines represent hazard ratios and 95% confidence intervals, respectively.

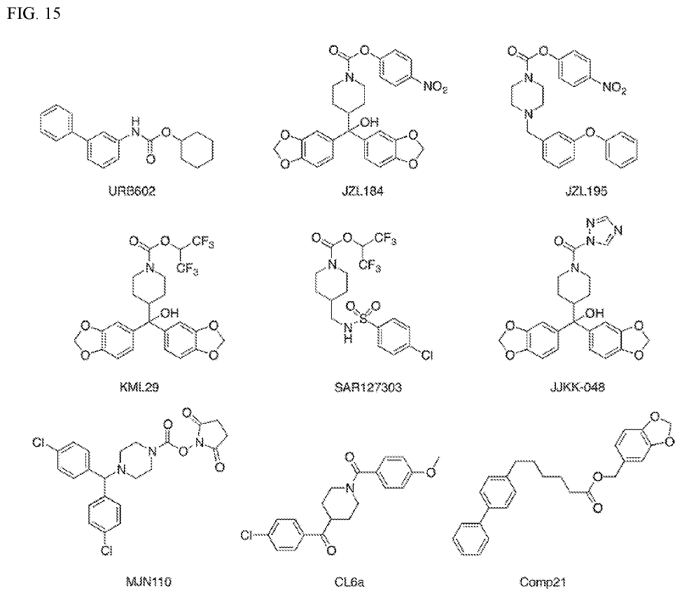

FIG. 15. Select examples of monoglyceride lipase (MGL) inhibitors.

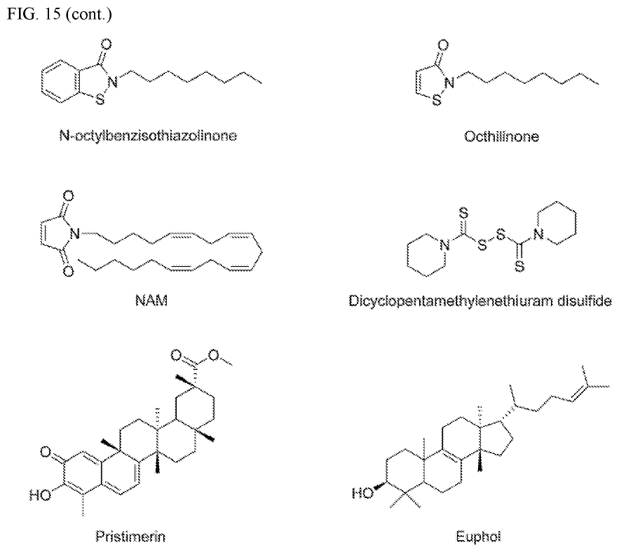

### DETAILED DESCRIPTION OF THE INVENTION

### A. Definitions

The abbreviations used herein have their conventional meaning within the chemical and biological arts.

Compound provided herein may be agents (e.g. compounds, proteins, drugs, detectable agents, therapeutic agents) in a prodrug form. Prodrugs of the compounds described herein are those compounds that readily undergo chemical changes under select physiological conditions to provide the final agents (e.g. compounds, proteins, drugs, detectable agents, therapeutic agents).

The terms “a” or “an,” as used in herein means one or more.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat kidney disease (e.g., chronic kidney disease, renal disease, end stage renal disease, end stage kidney disease). For example certain methods herein treat kidney disease (e.g., chronic kidney disease, renal disease, end stage renal disease, end stage kidney disease) by decreasing a symptom of kidney disease (e.g., chronic kidney disease, renal disease, end stage renal disease, end stage kidney disease). Symptoms of kidney disease (e.g., chronic kidney disease, renal disease, end stage renal disease, end stage kidney disease) would be known or may be determined by a person of ordinary skill in the art. The term “treating” and conjugations thereof, include prevention of an injury, pathology, condition, or disease. For example, certain methods herein treat kidney disease (e.g., chronic kidney disease, renal disease, end stage renal disease, end stage kidney disease). In embodiments, treating does not include preventing. For example, certain methods herein treat kidney disease (e.g., chronic kidney disease, renal disease, end stage renal disease, end stage kidney disease) by preventing a symptom (e.g., complication) of kidney disease (e.g., chronic kidney disease, renal disease, end stage renal disease, end stage kidney disease), for example wasting or cachexia.

An “effective amount” is an amount sufficient to accomplish a stated purpose (e.g. achieve the effect for which it is administered, treat a disease, reduce enzyme activity, increase enzyme activity, reduce protein function, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug or prodrug is an amount of a drug or prodrug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. kidney disease (e.g., chronic kidney disease, renal disease, end stage renal disease, end stage kidney disease)) means that the disease (e.g. kidney disease (e.g., chronic kidney disease, renal disease, end stage renal disease, end stage kidney disease)) is caused by (in whole or in part), or a symptom of the disease is caused by (in whole or in part) the substance or substance activity or function. As used herein, what is described as being associated with a disease, if a causative agent, could be a target for treatment of the disease.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

The term “modulator” refers to a composition that increases or decreases the level of a target molecule or the function of a target molecule. In embodiments, a modulator increases the level of activity of a cannabinoid receptor. In embodiments, a modulator decreases the level of activity of a cannabinoid receptor.

As defined herein, the term “activation”, “activate”, “activating” and the like in reference to a protein refers to conversion of a protein into a biologically active derivative from an initial inactive or deactivated state or increasing the level of activity of a target compared to control (e.g., absence of the activating agent). The terms reference activation, or activating, sensitizing, or up-regulating signal transduction or enzymatic activity or the amount of a protein decreased in a disease.

“Patient” or “subject in need thereof” or “subject” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In some embodiments, a patient is human. In some embodiments, a subject is human.

“Disease” or “condition” refer to a state of being or health status of a patient or subject capable of being treated with a compound, pharmaceutical composition, or method provided herein. In some embodiments, the disease is a disease having the symptom of reduced kidney function relative to normal kidney function in a subject (e.g. human). In some embodiments, the disease is kidney disease (e.g., chronic kidney disease, renal disease, end stage renal disease, end stage kidney disease). In some further instances, “kidney disease” refers to human kidney disease (e.g., chronic kidney disease, renal disease, end stage renal disease, end stage kidney disease).

As used herein, the term “kidney disease” or “renal disease” refers to a disease or condition related to reduction in kidney function compared to healthy kidney function.

As used herein, the terms “chronic kidney disease” or “chronic renal disease” refers to a disease or condition related to the progressive reduction in kidney function compared to healthy kidney function. Chronic kidney disease may be characterized by a glomerular filtration rate (GFR) of less than 90 ml/min/1.73 m² for three or more months or kidney damage (e.g., presence of high levels of protein in the urine, such as albumin). In embodiments, chronic kidney disease is stage 1 wherein glomerular filtration rate (GFR) is 90-120 ml/min/1.73 m² but there is radiologic or other evidence of kidney disease (such as protein in the urine). In embodiments, chronic kidney disease is stage 2 wherein glomerular filtration rate (GFR) is from 60 to 89 ml/min/1.73 m². In embodiments, chronic kidney disease is stage 3A wherein glomerular filtration rate (GFR) is from 45 to 59 ml/min/1.73 m². In embodiments, chronic kidney disease is stage 3B wherein glomerular filtration rate (GFR) is from 30 to 44 ml/min/1.73 m². In embodiments, chronic kidney disease is stage 4 wherein glomerular filtration rate (GFR) is from 15 to 29 ml/min/1.73 m². In embodiments, chronic kidney disease is stage 5 wherein glomerular filtration rate (GFR) is less than 15 ml/min/1.73 m², which is also called “end stage renal disease” or ESRD. A normal (e.g. healthy) glomerular filtration rate may be greater than or equal to 90 ml/min/1.73 m². A normal (e.g. healthy) glomerular filtration rate may be 90 to 120 ml/min/1.73 m². In embodiments, an average normal GFR (e.g., not associated with chronic kidney disease) associated with age (age in years: GFR) is 20-29:116, 30-39:107, 40-49:99, 50-59:93, 60-69:85, greater than 70:75.

As used herein, the term “end stage renal disease” or “ESRD” refers to kidney disease or chronic kidney disease (CDK) characterized by a glomerular filtration rate (GFR) of less than 15 ml/min/1.73 m². ESRD is also known as established renal failure. ESRD may be characterized by a glomerular filtration rate (GFR) of less than 10 ml/min/1.73 m². ESRD may be characterized by a glomerular filtration rate (GFR) of less than 5 ml/min/1.73 m². ESRD may be characterized by kidney function (e.g., filtration of waste and/or water from the blood) incapable of meeting the requirements of the body. ESRD may be characterized by less than 10% of normal (e.g. healthy) kidney function). Treatments for end stage renal disease include hemodialysis, peritoneal dialysis, home hemodialysis, and transplantation (e.g., kidney transplant).

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent, protein). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. The compositions of the present invention can also be delivered as nanoparticles.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “cannabinoid receptor” refers to a protein (including homologs, isoforms, and functional fragments thereof) that is a G protein-coupled receptor in the endocannabinoid system. The term includes any recombinant or naturally-occurring form of a cannabinoid receptor or variants thereof that maintain cannabinoid receptor activity (e.g. within at least 30%, 40%, 50%, 60%, 70%, 80%, 90%, 95%, or 100% activity compared to wildtype cannabinoid receptor). In embodiments, the cannabinoid receptor protein is cannabinoid receptor 1 and is encoded by the CNR1 gene has the amino acid sequence set forth in or corresponding to Entrez 1268, UniProt P21554, or RefSeq (protein) NP 057167. In embodiments, the cannabinoid receptor protein 1 gene has the nucleic acid sequence set forth in RefSeq (mRNA) NM_016083. In embodiments, the amino acid sequence or nucleic acid sequence is the sequence known at the time of filing of the present application. In embodiments, the sequence corresponds to NP_057167.2. In embodiments, the sequence corresponds to NM_016083.4. In embodiments, the cannabinoid receptor protein 1 is a human cannabinoid receptor protein 1. In embodiments, the cannabinoid receptor protein is cannabinoid receptor 2 and is encoded by the CNR2 gene has the amino acid sequence set forth in or corresponding to Entrez 1269, UniProt P34972, or RefSeq (protein) NP_001832. In embodiments, the cannabinoid receptor protein 2 gene has the nucleic acid sequence set forth in RefSeq (mRNA) NM_001841. In embodiments, the amino acid sequence or nucleic acid sequence is the sequence known at the time of filing of the present application. In embodiments, the sequence corresponds to NP_001832.1. In embodiments, the sequence corresponds to NM_001841.2. In embodiments, the cannabinoid receptor protein 2 is a human cannabinoid receptor protein 2.

The term “monoacylglycerol lipase”, “MAG lipase”, “MAGL”, “MGL”, or “MGLL” refers to a protein (including homologs, isoforms, and functional fragments thereof) that, in humans, is encoded by the MGLL gene. MGL is a 33-kDa, membrane-associated member of the serine hydrolase superfamily and contains the classical GXSXG consensus sequence common to most serine hydrolases, wherein X may be any residue. The catalytic triad has been identified as Ser122, His269, and Asp239. In embodiments, monoacylglycerol lipase has the amino acid sequence set forth in or corresponding to Entrez 11343, UniProt Q99685, or RefSeq (protein) NP_009214. In embodiments, monoacylglycerol lipase has the nucleic acid sequence set forth in RefSeq (mRNA) NM_007283. In embodiments, the amino acid sequence or nucleic acid sequence is the sequence known at the time of filing of the present application. In embodiments, the sequence corresponds to NP_009214.1. In embodiments, the sequence corresponds to NM_007283.6.

The term “tetrahydrocannabinol” or “THC” refers to a cannabinoid that is present in cannabis. THC is the principal psychoactive constituent of cannabis. The chemical name of THC is (−)-trans-Δ⁹-tetrahydrocannabinol or (6aR,10aR)-delta-9-tetrahydrocannabinol. In embodiments, the term THC also refers to cannabinoid isomers.

The term “allosteric modulator” refers to a substance which indirectly influences (modulates) the effects of a primary ligand that directly activates or deactivates the function of a target protein. Targets may be metabotropic, ionotropic and nuclear receptors, enzymes and transporters. The term “allosteric modulator of a cannabinoid receptor” refers to a substance which indirectly influences (modulates) the effects of a primary ligand that directly activates or deactivates the function of a cannabinoid receptor. The term “positive allosteric modulator”, “PAM”, “allosteric enhancer” or “allosteric potentiator”, refers to an allosteric modulator that induces an amplification of the effect of receptor's response to the primary ligand without directly activating the receptor. The term “pan positive allosteric modulator of a cannabinoid receptor”, refers to a positive allosteric modulator that modulates all cannabinoid receptors, including cannabinoid receptor type 1 and cannabinoid receptor type 2.

### B. Methods

In an aspect is provided a method of treating chronic kidney disease in a subject in need thereof, the method including administering an effective amount of an agent that increases the level of activity of a cannabinoid receptor, to the subject.

In embodiments, the cannabinoid receptor is human cannabinoid receptor type 1. In embodiments, the agent is an agonist of a cannabinoid receptor. In embodiments, the agent (e.g., agonist) is anandamide or a derivative thereof, tetrahydrocannabinol or a derivative thereof, 2-arachidonoyl-sn-glycerol (2-AG) or a derivative thereof, cannabidiol, or cannabis extract. In embodiments, the agent (e.g., agonist) is anandamide, tetrahydrocannabinol, 2-arachidonoyl-sn-glycerol (2-AG), cannabidiol, or cannabis extract. In embodiments, the agent (e.g., agonist) is 2-arachidonoyl-sn-glycerol (2-AG). In embodiments, the agent (e.g., agonist) is tetrahydrocannabinol or a derivative thereof, 2-arachidonoyl-sn-glycerol (2-AG) or a derivative thereof, cannabidiol, or a derivative thereof, or cannabis extract or a derivative thereof. In embodiments, the agent (e.g., agonist) is tetrahydrocannabinol, 2-arachidonoyl-sn-glycerol (2-AG), cannabidiol, or cannabis extract. In embodiments, the agent (e.g., agonist) is 2-arachidonoyl-sn-glycerol (2-AG). In embodiments, the agent inhibits the degradation of an agonist of a cannabinoid receptor. In embodiments, the agent is an inhibitor of monoacylglycerol lipase (MGL). In embodiments, the agent (e.g., agonist) is tetrahydrocannabinol or a derivative thereof. In embodiments, the agent (e.g., agonist) is 2-arachidonoyl-sn-glycerol (2-AG) or a derivative thereof. In embodiments, the agent (e.g., agonist) is cannabidiol or a derivative thereof. In embodiments, the agent (e.g., agonist) is cannabis extract or a derivative thereof. In embodiments, the agent (e.g., agonist) is tetrahydrocannabinol. In embodiments, the agent (e.g., agonist) is 2-arachidonoyl-sn-glycerol (2-AG), In embodiments, the agent (e.g., agonist) is cannabidiol. In embodiments, the agent (e.g., agonist) is cannabis extract.

In embodiments, the agent is an activator of a cannabinoid receptor. In embodiments, the agent is an activator of cannabinoid receptor type 1. In embodiments, the agent is a pan positive allosteric modulator of a cannabinoid receptor. In embodiments, the agent is a positive allosteric modulator of a cannabinoid receptor. In embodiments, agent is a synthetic positive allosteric modulator of a cannabinoid receptor. In embodiments, agent is a positive allosteric modulator of cannabinoid receptor type 1.

In embodiments, the agent is URB602 (cyclohexyl [1,1′-biphenyl]-3-ylcarbamate), URB754 (6-methyl-2-[(4-methylphenyl)amino]-4H-3,1-benzoxazin-4-one), MGL184, N-arachidonoyl maleimide (NAM), JZL184 (4-nitrophenyl-4-(dibenzo[d][1,3]dioxol-5-yl(hydroxy)methyl)piperidine-1-carboxylate), JZL195 ((4-nitrophenyl) 4-[(3-phenoxyphenyl)methyl]piperazine-1-carboxylate), JNJ-42165279 (N-(4-chloropyridin-3-yl)-4-[(2,2-difluoro-1,3-benzodioxol-5-yl)methyl]piperazine-1-carboxamide), JW 642 (4-[(3-Phenoxyphenyl)methyl]-1-piperazinecarboxylic acid 2,2,2-trifluoro-1-(trifluoromethyl)ethyl ester), KML29 (1,1,1,3,3,3-hexafluoropropan-2-yl 4-(bis(benzo[d][1,3]dioxol-5-yl)(hydroxy)methyl)piperidine-1-carboxylate), SAR127303 (1,1,1,3,3,3-hexafluoropropan-2-yl 4-(((4-chlorophenyl)sulfonamido)methyl)piperidine-1-carboxylate), JJKK-048 (4-[Bis(1,3-benzodioxol-5-yl)methyl]-1-piperidinyl]-1H-1,2,4-triazol-1-yl-methanone), MJN110 (2,5-dioxopyrrolidin-1-yl 4-(bis(4-chlorophenyl)methyl)piperazine-1-carboxylate), CL6a ((4-(4-chlorobenzoyl)piperidin-1-yl)(4-methoxyphenyl)methanone), Comp21 (benzo[d][1,3]dioxol-5-ylmethyl 6-([1,1′-biphenyl]-4-yl)hexanoate), N-octylbenzisothiazolinone, octhilinone (2-octylisothiazol-3(2H)-one), dicyclopentamethylenethiuram disulfide, pristimerin, or euphol. In embodiments, the agent is URB602 (cyclohexyl [1,1′-biphenyl]-3-ylcarbamate), URB754 (6-methyl-2-[(4-methylphenyl)amino]-4H-3,1-benzoxazin-4-one), N-arachidonoyl maleimide (NAM), JZL184 (4-nitrophenyl-4-(dibenzo[d][1,3]dioxol-5-yl(hydroxy)methyl)piperidine-1-carboxylate), JZL195 ((4-nitrophenyl) 4-[(3-phenoxyphenyl)methyl]piperazine-1-carboxylate), JNJ-42165279 (N-(4-chloropyridin-3-yl)-4-[(2,2-difluoro-1,3-benzodioxol-5-yl)methyl]piperazine-1-carboxamide), JW 642 (4-[(3-Phenoxyphenyl)methyl]-1-piperazinecarboxylic acid 2,2,2-trifluoro-1-(trifluoromethyl)ethyl ester), KML29 (1,1,1,3,3,3-hexafluoropropan-2-yl 4-(bis(benzo[d][1,3]dioxol-5-yl)(hydroxy)methyl)piperidine-1-carboxylate), SAR127303 (1,1,1,3,3,3-hexafluoropropan-2-yl 4-(((4-chlorophenyl)sulfonamido)methyl)piperidine-1-carboxylate), JJKK-048 (4-[Bis(1,3-benzodioxol-5-yl)methyl]-1-piperidinyl]-1H-1,2,4-triazol-1-yl-methanone), MJN110 (2,5-dioxopyrrolidin-1-yl 4-(bis(4-chlorophenyl)methyl)piperazine-1-carboxylate), CL6a ((4-(4-chlorobenzoyl)piperidin-1-yl)(4-methoxyphenyl)methanone), Comp21 (benzo[d][1,3]dioxol-5-ylmethyl 6-([1,1′-biphenyl]-4-yl)hexanoate), N-octylbenzisothiazolinone, octhilinone (2-octylisothiazol-3(2H)-one), dicyclopentamethylenethiuram disulfide, pristimerin, or euphol. In embodiments, the agent is URB602, or a derivative thereof. In embodiments, the agent is URB754 (cyclohexyl [1,1′-biphenyl]-3-ylcarbamate), or a derivative thereof. In embodiments, the agent is MGL184, or a derivative thereof. In embodiments, the agent is N-arachidonoyl maleimide (NAM), or a derivative thereof. In embodiments, the agent is JZL184 (4-nitrophenyl-4-(dibenzo[d][1,3]dioxol-5-yl(hydroxy)methyl)piperidine-1-carboxylate), or a derivative thereof. In embodiments, the agent is JZL195 ((4-nitrophenyl) 4-[(3-phenoxyphenyl)methyl]piperazine-1-carboxylate), or a derivative thereof. In embodiments, the agent is JNJ-42165279 (N-(4-chloropyridin-3-yl)-4-[(2,2-difluoro-1,3-benzodioxol-5-yl)methyl]piperazine-1-carboxamide), or a derivative thereof. In embodiments, the agent is JW 642 (4-[(3-Phenoxyphenyl)methyl]-1-piperazinecarboxylic acid 2,2,2-trifluoro-1-(trifluoromethyl)ethyl ester), or a derivative thereof In embodiments, the agent is KML29 (1,1,1,3,3,3-hexafluoropropan-2-yl 4-(bis(benzo[d][1,3]dioxol-5-yl)(hydroxy)methyl)piperidine-1-carboxylate), or a derivative thereof. In embodiments, the agent is SAR127303 (1,1,1,3,3,3-hexafluoropropan-2-yl 4-(((4-chlorophenyl)sulfonamido)methyl)piperidine-1-carboxylate), or a derivative thereof. In embodiments, the agent is JJKK-048 (4-[Bis(1,3-benzodioxol-5-yl)methyl]-1-piperidinyl]-1H-1,2,4-triazol-1-yl-methanone), or a derivative thereof. In embodiments, the agent is MJN110 (2,5-dioxopyrrolidin-1-yl 4-(bis(4-chlorophenyl)methyl)piperazine-1-carboxylate), or a derivative thereof. In embodiments, the agent is CL6a ((4-(4-chlorobenzoyl)piperidin-1-yl)(4-methoxyphenyl)methanone), or a derivative thereof. In embodiments, the agent is Comp21 (benzo[d][1,3]dioxol-5-ylmethyl 6-([1,1′-biphenyl]-4-yl)hexanoate), or a derivative thereof. In embodiments, the agent is N-octylbenzisothiazolinone, or a derivative thereof. In embodiments, the agent is octhilinone, or a derivative thereof. In embodiments, the agent is dicyclopentamethylenethiuram disulfide, or a derivative thereof. In embodiments, the agent is pristimerin, or a derivative thereof. In embodiments, the agent is euphol, or a derivative thereof.

In embodiments, the agent is URB602 (cyclohexyl [1,1′-biphenyl]-3-ylcarbamate), URB754 (6-methyl-2-[(4-methylphenyl)amino]-4H-3,1-benzoxazin-4-one), MGL184, N-arachidonoyl maleimide (NAM), JZL184 (4-nitrophenyl-4-(dibenzo[d][1,3]dioxol-5-yl(hydroxy)methyl)piperidine-1-carboxylate), JZL195 ((4-nitrophenyl) 4-[(3-phenoxyphenyl)methyl]piperazine-1-carboxylate), KML29 (1,1,1,3,3,3-hexafluoropropan-2-yl 4-(bis(benzo[d][1,3]dioxol-5-yl)(hydroxy)methyl)piperidine-1-carboxylate), SAR127303 (1,1,1,3,3,3-hexafluoropropan-2-yl 4-(((4-chlorophenyl)sulfonamido)methyl)piperidine-1-carboxylate), JJKK-048 (4-[Bis(1,3-benzodioxol-5-yl)methyl]-1-piperidinyl]-1H-1,2,4-triazol-1-yl-methanone), MJN110 (2,5-dioxopyrrolidin-1-yl 4-(bis(4-chlorophenyl)methyl)piperazine-1-carboxylate), CL6a ((4-(4-chlorobenzoyl)piperidin-1-yl)(4-methoxyphenyl)methanone), or Comp21 (benzo[d][1,3]dioxol-5-ylmethyl 6-([1,1′-biphenyl]-4-yl)hexanoate). In embodiments, the agent is URB602 (cyclohexyl [1,1′-biphenyl]-3-ylcarbamate), URB754 (6-methyl-2-[(4-methylphenyl)amino]-4H-3,1-benzoxazin-4-one), N-arachidonoyl maleimide (NAM), JZL184 (4-nitrophenyl-4-(dibenzo[d][1,3]dioxol-5-yl(hydroxy)methyl)piperidine-1-carboxylate), JZL195 ((4-nitrophenyl) 4-[(3-phenoxyphenyl)methyl]piperazine-1-carboxylate), KML29 (1,1,1,3,3,3-hexafluoropropan-2-yl 4-(bis(benzo[d][1,3]dioxol-5-yl)(hydroxy)methyl)piperidine-1-carboxylate), SAR127303 (1,1,1,3,3,3-hexafluoropropan-2-yl 4-(((4-chlorophenyl)sulfonamido)methyl)piperidine-1-carboxylate), JJKK-048 (4-[Bis(1,3-benzodioxol-5-yl)methyl]-1-piperidinyl]-1H-1,2,4-triazol-1-yl-methanone), MJN110 (2,5-dioxopyrrolidin-1-yl 4-(bis(4-chlorophenyl)methyl)piperazine-1-carboxylate), CL6a ((4-(4-chlorobenzoyl)piperidin-1-yl)(4-methoxyphenyl)methanone), or Comp21 (benzo[d][1,3]dioxol-5-ylmethyl 6-([1,1′-biphenyl]-4-yl)hexanoate). In embodiments, the agent is N-octylbenzisothiazolinone, octhilinone, dicyclopentamethylenethiuram disulfide, pristimerin, or euphol. In embodiments, the agent is URB602 (cyclohexyl [1,1′-biphenyl]-3-ylcarbamate), URB754 (6-methyl-2-[(4-methylphenyl)amino]-4H-3,1-benzoxazin-4-one), MGL184, N-arachidonoyl maleimide (NAM), or JZL184 (4-nitrophenyl-4-(dibenzo[d][1,3]dioxol-5-yl(hydroxy)methyl)piperidine-1-carboxylate). In embodiments, the agent is URB602 (cyclohexyl [1,1′-biphenyl]-3-ylcarbamate), URB754 (6-methyl-2-[(4-methylphenyl)amino]-4H-3,1-benzoxazin-4-one), N-arachidonoyl maleimide (NAM), or JZL184 (4-nitrophenyl-4-(dibenzo[d][1,3]dioxol-5-yl(hydroxy)methyl)piperidine-1-carboxylate). In embodiments, the agent is THC. In embodiments, the agent is THC or a derivative thereof. In embodiments, the agent is (−)-trans-Δ⁹-tetrahydrocannabinol. In embodiments, the agent is (−)-trans-Δ⁹-tetrahydrocannabinol, or a derivative thereof. In embodiments, the agent is cannabidiol. In embodiments, the agent is cannabidiol, or a derivative thereof. In embodiments, the agent is cannabis extract. In embodiments, the agent is cannabis extract, or a derivative thereof.

In an aspect is provided a method of treating chronic kidney disease in a subject in need thereof, the method including administering an effective amount of an agent that increases the serum level of 2-arachidonoyl-sn-glycerol (2-AG), to the subject.

In an aspect is provided a method of treating chronic kidney disease in a subject in need thereof, the method including administering an effective amount of an agent that increases the tissue (e.g., renal) level of 2-arachidonoyl-sn-glycerol (2-AG), to the subject.

In embodiments, the agent is 2-arachidonoyl-sn-glycerol (2-AG). In embodiments, the agent reduces the degradation of 2-arachidonoyl-sn-glycerol (2-AG). In embodiments, the agent is an inhibitor of monoacylglycerol lipase (MGL). In embodiments, the agent is URB602, MGL184, N-arachidonoyl maleimide, or JZL184 (4-nitrophenyl-4-(dibenzo[d][1,3]dioxol-5-yl(hydroxy)methyl)piperidine-1-carboxylate). In embodiments, the agent is URB602, N-arachidonoyl maleimide, or JZL184 (4-nitrophenyl-4-(dibenzo[d][1,3]dioxol-5-yl(hydroxy)methyl)piperidine-1-carboxylate). In embodiments, the agent is a precursor in the biosynthesis of 2-arachidonoyl-sn-glycerol (2-AG). In embodiments, the agent is 1-palmitoyl-2-arachidonoyl-sn-glycerol. In embodiments, the serum level of 2-arachidonoyl-sn-glycerol (2-AG) is increased in the subject to greater than about 117.16 pmol/mL. In embodiments, the serum level of 2-arachidonoyl-sn-glycerol (2-AG) is increased in the subject to greater than 117.16 pmol/mL. In embodiments, the serum level of 2-arachidonoyl-sn-glycerol (2-AG) is increased in the subject to greater than about 10, 20, 30, 40, 50, 60, 70, 80, 90, 100, 110, 120, 130, 140, 150, 160, 170, 180, 190, 200, 250, 300, 350, 400, 450, 500, 600, 700, 800, 900, or 1000 pmol/mL. In embodiments, the serum level of 2-arachidonoyl-sn-glycerol (2-AG) is about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, or 185 pmol/mL.

In embodiments, chronic kidney disease is end stage renal disease. In embodiments, the subject has cachexia. In embodiments, the subject has protein energy wasting (PEW). In embodiments, the subject is being treated with maintenance hemodialysis. In embodiments, treating chronic kidney disease (e.g., end stage renal disease) is increasing survival (e.g., compared to control, such as in the absence of treatment). In embodiments, treating chronic kidney disease (e.g., end stage renal disease) is extending time of survival following treatment (e.g., compared to control, such as in the absence of treatment). In embodiments, the extension of time of survival is at least 1, 2, 3, 4, 5, 6, 7, 8, 9, 10, 11, 12, 13, 14, 15, 16, 17, 18, 19, 20, 21, 22, 23, or 24 days. In embodiments, the extension of time of survival is at least 1, 2, 3, 4, 5, 6, 7, 8, 9, 10, 11, 12, 13, 14, 15, 16, 17, 18, 19, 20, 21, 22, 23, or 24 weeks. In embodiments, the extension of time of survival is at least 1, 2, 3, 4, 5, 6, 7, 8, 9, 10, 11, 12, 13, 14, 15, 16, 17, 18, 19, 20, 21, 22, 23, or 24 months. In embodiments, the extension of time of survival is at least 1, 2, 3, 4, 5, 6, 7, 8, 9, or 10 years. In embodiments, treating kidney disease (e.g., chronic kidney disease, renal disease, end stage renal disease, end stage kidney disease) includes preventing a symptom (e.g, complication) of kidney disease (e.g., chronic kidney disease, renal disease, end stage renal disease, end stage kidney disease). In embodiments a symptom (e.g, complication) of kidney disease (e.g., chronic kidney disease, renal disease, end stage renal disease, end stage kidney disease) includes wasting or cachexia.

In embodiments, the route of administration is intraperitoneal administration. In embodiments, the route of administration is as a suppository. In embodiments, the route of administration is topical. In embodiments, the route of administration is intravenous. In embodiments, the route of administration is parenteral. In embodiments, the route of administration is intraperitoneal. In embodiments, the route of administration is intramuscular. In embodiments, the route of administration is intralesional. In embodiments, the route of administration is intrathecal. In embodiments, the route of administration is intracranial. In embodiments, the route of administration is intranasal. In embodiments, the route of administration is subcutaneous. In embodiments, the route of administration is oral. In embodiments, the route of administration is sublingual. In embodiments, the route of administration is inhalation. In embodiments, the route of administration is inhalation by using a vaporizer. In embodiments, the route of administration is a vape pen. In embodiments, the route of administration is a gel capsule. In embodiments, the route of administration is a snuff pack. In embodiments, the route of administration is a troche.

In embodiments, the marker used to measure improved kidney function, reduced cachexia, or reduced wasting, is: reduced systolic blood pressure, decreased rate of urine protein excretion, improved renal function, improvement in blood pressure, reduced serum BUN concentration, decreased urinary protein excretion, or reduced metabolic rate. In embodiments, the marker used to measure improved kidney function, reduced cachexia, or reduced wasting is reduced systolic blood pressure. In embodiments, the marker used to measure improved kidney function, reduced cachexia, or reduced wasting is decreased rate of urine protein excretion. In embodiments, the marker used to measure improved kidney function, reduced cachexia, or reduced wasting is improved renal function. In embodiments, the marker used to measure improved kidney function, reduced cachexia, or reduced wasting is improvement in blood pressure. In embodiments, the marker used to measure improved kidney function, reduced cachexia, or reduced wasting is reduced serum BUN concentration. In embodiments, the marker used to measure improved kidney function, reduced cachexia, or reduced wasting is decreased urinary protein excretion. In embodiments, the marker used to measure improved kidney function, reduced cachexia, or reduced wasting is reduced metabolic rate. In embodiments, the marker used to measure improved kidney function is improvement (e.g., reduction) in blood pressure, decreased urine protein excretion, or reduced serum blood urea nitrogen (BUN). In embodiments, the marker used to measure improved kidney function is improvement in blood pressure. In embodiments, the marker used to measure improved kidney function is decreased urine protein excretion. In embodiments, the marker used to measure improved kidney function is decreased serum blood urea nitrogen (BUN). In embodiments, the marker used to measure reduced cachexia or reduced wasting is increased body mass or increased muscle mass. In embodiments, the marker used to measure reduced cachexia is increased body mass. In embodiments, the marker used to measure reduced cachexia is increased muscle mass. In embodiments, the marker used to measure reduced wasting is increased body mass. In embodiments, the marker used to measure reduced wasting is increased muscle mass. In embodiments, increased muscle mass is measured by mid arm circumference or tricep circumference.

In embodiments, metabolic rate determinations are made using the TSE PhenoMaster System. In embodiments, test for measuring metabolic rate is a basal metabolic rate (BMR) test, resting metabolic rate (RMR) test or an exercise test. In embodiments, the RMR test is a direct calorimetry test. In embodiments, the RMR test is an indirect calorimetry test. In embodiments, metabolic rate determinations are made by measuring metabolic rate in humans. In embodiments, metabolic rate determinations are made by measuring metabolic rate in mice. In embodiments, the metabolic rate of mice is determined by measuring CO₂ production or O₂ consumption. In embodiments, the metabolic rate of mice is determined by measuring or calculating the respiratory quotient or energy expenditure. In embodiments, reducing metabolic rate reduces the risk of cachexia. In embodiments, reducing metabolic rate reduces the risk of wasting. In embodiments, reducing metabolic rate reduces the risk of muscle wasting. In embodiments, increasing tissue 2-AG levels reduces the risk of cachexia. In embodiments, increasing tissue 2-AG levels reduces the risk of wasting. In embodiments, increasing tissue 2-AG levels reduces the risk of muscle wasting. In embodiments, reducing metabolic rate reduces cachexia. In embodiments, reducing metabolic rate reduces wasting. In embodiments, reducing metabolic rate reduces muscle wasting. In embodiments, increasing tissue 2-AG levels reduces cachexia. In embodiments, increasing tissue 2-AG levels reduces wasting. In embodiments, increasing tissue 2-AG levels reduces muscle wasting.

In embodiments, is a method of treating chronic kidney disease, end stage renal disease, wasting or cachexia by increasing the levels of 2-AG. In embodiments, the 2-AG levels are increased in the brain. In embodiments, the 2-AG levels are increased in the kidney. In embodiments, the 2-AG levels are increased in the fat. In embodiments, the levels of brown fat are reduced. In embodiments, the levels of white fat are increased. In embodiments, the weight of the subject is increased. In embodiments, the percent body fat of the subject is increased. In embodiments, the BMI of the subject is increased. In embodiments, the BMI is increased above a level of 25 kg/m². In embodiments, the BMI is increased above a level of 27 kg/m². In embodiments, the BMI is increased above a level of 30 kg/m². In embodiments, the level of serum triglycerides is increased. In embodiments, the level of serum triglycerides is increased above a level of 126 mg/dL. In embodiments, the level of serum triglycerides is increased above a level of 160 mg/dL. In embodiments, the ratio of brown fat to white fat in a patient is decreased.

In an aspect is provided a method of identifying a subject for treatment with a method described herein, including detecting the serum level of 2-arachidonoyl-sn-glycerol (2-AG) in a candidate subject. In embodiments, the serum level of 2-arachidonoyl-sn-glycerol (2-AG) in a candidate subject is less than control (e.g., control is a healthy person or a person who would not benefit from a method described herein). In embodiments, the candidate subject is identified as a subject by detection of a serum level of 2-arachidonoyl-sn-glycerol (2-AG) less than control (e.g., control is a healthy person or a person who would not benefit from a method described herein). In embodiments, the candidate subject is identified as a subject by detection of a serum level of 2-arachidonoyl-sn-glycerol (2-AG) less than 117.16 pmol/mL. In embodiments, the candidate subject is identified as a subject by detection of a serum level of 2-arachidonoyl-sn-glycerol (2-AG) less than about 117.16 pmol/mL. In embodiments, the candidate subject is identified as a subject by detection of a serum level of 2-arachidonoyl-sn-glycerol (2-AG) less than 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, or 185 pmol/mL. In embodiments, the candidate subject is identified as a subject by detection of a serum level of 2-arachidonoyl-sn-glycerol (2-AG) less than about 55.97 pmol/mL in the subject. In embodiments, the level of 2-arachidonoyl-sn-glycerol (2-AG) is less than 55.97 pmol/mL in the subject. In embodiments, the level of 2-arachidonoyl-sn-glycerol (2-AG) is less than about 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500 600, 700, 800, 900, or 1000 pmol/mL in the subject. In embodiments, the method is a method of identifying a subject for treatment with a method described herein, including detecting the serum level of 2-arachidonoyl-sn-glycerol (2-AG) in a candidate subject; wherein the candidate subject is identified as a subject by detection of a serum level of 2-arachidonoyl-sn-glycerol (2-AG) less than 55.97 pmol/mL in the subject.

### C. Embodiments

Embodiment P1. A method of treating chronic kidney disease in a subject in need thereof, the method comprising administering an effective amount of an agent that increases the level of activity of a cannabinoid receptor to the subject.

Embodiment P2. The method of embodiment P1, wherein the cannabinoid receptor is human cannabinoid receptor type 1.

Embodiment P3. The method of one of embodiments P1 to P2, wherein the agent is an agonist of a cannabinoid receptor.

Embodiment P4. The method of embodiment P3, wherein the agonist is anandamide or a derivative thereof, tetrahydrocannabinol or a derivative thereof, 2-arachidonoyl-sn-glycerol (2-AG) or a derivative thereof, cannabidiol, or cannabis extract.

Embodiment P5. The method of embodiment P3, wherein the agonist is 2-arachidonoyl-sn-glycerol (2-AG).

Embodiment P6. The method of one of embodiments P1 to P2, wherein the agent inhibits the degradation of an agonist of a cannabinoid receptor.

Embodiment P7. The method of embodiment P6, wherein the agent is an inhibitor of monoacylglycerol lipase (MGL).

Embodiment P8. The method of embodiment P6, wherein the agent is URB602, MGL184, N-arachidonoyl maleimide, or JZL184 (4-nitrophenyl-4-(dibenzo[d][1,3]dioxol-5-yl(hydroxy)methyl)piperidine-1-carboxylate).

Embodiment P9. A method of treating chronic kidney disease in a subject in need thereof, the method comprising administering an effective amount of an agent that increases the serum level of 2-arachidonoyl-sn-glycerol (2-AG), to the subject.

Embodiment P10. The method of embodiment P9, wherein the agent is 2-arachidonoyl-sn-glycerol (2-AG).

Embodiment P11. The method of embodiment P9, wherein the agent reduces the degradation of 2-arachidonoyl-sn-glycerol (2-AG).

Embodiment P12. The method of embodiment P11, wherein the agent is an inhibitor of monoacylglycerol lipase (MGL).

Embodiment P13. The method of embodiment P12, wherein the agent is URB602, MGL184, N-arachidonoyl maleimide, or JZL184 (4-nitrophenyl-4-(dibenzo[d][1,3]dioxol-5-yl(hydroxy)methyl)piperidine-1-carboxylate).

Embodiment P14. The method of embodiment P9, wherein the agent is a precursor in the biosynthesis of 2-arachidonoyl-sn-glycerol (2-AG).

Embodiment P15. The method of embodiment P14, wherein the agent is 1-palmitoyl-2-arachidonoyl-sn-glycerol.

Embodiment P16. The method of one of embodiments P9 to P15, wherein the serum level of 2-arachidonoyl-sn-glycerol (2-AG) is increased in the subject is increased to greater than 117.16 pmol/mL.

Embodiment P17. The method of one of embodiments P1 to P16, wherein the chronic kidney disease is end stage renal disease.

Embodiment P18. The method of one of embodiments P1 to P17, wherein the subject has cachexia.

Embodiment P19. A method of identifying the subject of one of embodiments P1 to P18, comprising detecting the serum level of 2-arachidonoyl-sn-glycerol (2-AG) in a candidate subject; wherein the candidate subject is identified as a subject by detection of a serum level of 2-arachidonoyl-sn-glycerol (2-AG) less than 55.97 pmol/mL in the subject.

### D. Additional Embodiments

Embodiment 1. A method of treating chronic kidney disease in a subject in need thereof, the method comprising administering an effective amount of an agent that increases the level of activity of a cannabinoid receptor to the subject.

Embodiment 2. The method of embodiment 1, wherein the cannabinoid receptor is human cannabinoid receptor type 1.

Embodiment 3. The method of one of embodiments 1 to 2, wherein the agent is an agonist of a cannabinoid receptor.

Embodiment 4. The method of one of embodiments 1 to 3, wherein the agent is an agonist of human cannabinoid receptor type 1.

Embodiment 5. The method of one of embodiments 1 to 3, wherein the agent is an endocannabinoid.

Embodiment 6. The method of embodiment 3, wherein the agonist is anandamide or a derivative thereof, tetrahydrocannabinol or a derivative thereof, 2-arachidonoyl-sn-glycerol (2-AG) or a derivative thereof, cannabidiol or a derivative thereof, or cannabis extract.

Embodiment 7. The method of embodiment 3, wherein the agonist is 2-arachidonoyl-sn-glycerol (2-AG).

Embodiment 8. The method of one of embodiments 1 to 2, wherein the agent inhibits the degradation of an agonist of a cannabinoid receptor.

Embodiment 9. The method of embodiment 8, wherein the agent is an inhibitor of monoacylglycerol lipase (MGL).

Embodiment 10. The method of embodiment 8, wherein the agent is URB602, MGL184, N-arachidonoyl maleimide, JZL184 (4-nitrophenyl-4-(dibenzo[d][1,3]dioxol-5-yl(hydroxy)methyl)piperidine-1-carboxylate), JZL195, KML29, SAR127303, JJKK-048, MJN110, CL6a, Comp21, N-octylbenzisothiazolinone, octhilinone, NAM, dicyclopentamethylenethiuram disulfide, pristimerin, or euphol.

Embodiment 11. The method of embodiment 8, wherein the agent is URB602, MGL184, N-arachidonoyl maleimide, JZL184 (4-nitrophenyl-4-(dibenzo[d][1,3]dioxol-5-yl(hydroxy)methyl)piperidine-1-carboxylate), JZL195, KML29, SAR127303, JJKK-048, MJN110, CL6a, or Comp21.

Embodiment 12. The method of embodiment 8, wherein the agent is N-octylbenzisothiazolinone, octhilinone, NAM, dicyclopentamethylenethiuram disulfide, pristimerin, or euphol.

Embodiment 13. The method of embodiment 8, wherein the agent is URB602, MGL184, N-arachidonoyl maleimide, or JZL184 (4-nitrophenyl-4-(dibenzo[d][1,3]dioxol-5-yl(hydroxy)methyl)piperidine-1-carboxylate).

Embodiment 14. A method of treating chronic kidney disease in a subject in need thereof, the method comprising administering an effective amount of an agent that increases the serum level of 2-arachidonoyl-sn-glycerol (2-AG), to the subject.

Embodiment 15. The method of embodiment 14, wherein the agent is 2-arachidonoyl-sn-glycerol (2-AG).

Embodiment 16. The method of embodiment 14, wherein the agent reduces the degradation of 2-arachidonoyl-sn-glycerol (2-AG).

Embodiment 17. The method of embodiment 16, wherein the agent is an inhibitor of monoacylglycerol lipase (MGL).

Embodiment 18. The method of embodiment 17, wherein the agent is URB602, MGL184, N-arachidonoyl maleimide, JZL184 (4-nitrophenyl-4-(dibenzo[d][1,3]dioxol-5-yl(hydroxy)methyl)piperidine-1-carboxylate), JZL195, KML29, SAR127303, JJKK-048, MJN110, CL6a, Comp21, N-octylbenzisothiazolinone, octhilinone, NAM, dicyclopentamethylenethiuram disulfide, pristimerin, or euphol.

Embodiment 19. The method of embodiment 17, wherein the agent is URB602, MGL184, N-arachidonoyl maleimide, or JZL184 (4-nitrophenyl-4-(dibenzo[d][1,3]dioxol-5-yl(hydroxy)methyl)piperidine-1-carboxylate).

Embodiment 20. The method of embodiment 14, wherein the agent is a precursor in the biosynthesis of 2-arachidonoyl-sn-glycerol (2-AG).

Embodiment 21. The method of embodiment 20, wherein the agent is 1-palmitoyl-2-arachidonoyl-sn-glycerol.

Embodiment 22. The method of one of embodiments 14 to 21, wherein the serum level of 2-arachidonoyl-sn-glycerol (2-AG) is increased in the subject is increased to greater than 117.16 pmol/mL.

Embodiment 23. The method of one of embodiments 1 to 22, wherein the chronic kidney disease is end stage renal disease.

Embodiment 24. The method of one of embodiments 1 to 23, wherein the subject has cachexia.

Embodiment 25. A method of identifying the subject of one of embodiments 1 to 24, comprising detecting the serum level of 2-arachidonoyl-sn-glycerol (2-AG) in a candidate subject; wherein the candidate subject is identified as a subject by detection of a serum level of 2-arachidonoyl-sn-glycerol (2-AG) less than 55.97 pmol/mL in the subject.

### EXAMPLES

CKD is associated with a significantly increased risk of morbidity and mortality and this is especially pronounced in ESRD patients who experience a disproportionately elevated risk of death. Traditional risk factors for mortality in the non-ESRD population such as obesity and hypertriglyceridemia, do not consistently explain the mortality risk observed in these patients and in some cases, can be associated with improved outcomes. (4,5) However, these contradictory associations (i.e. higher BMI and increased serum TG levels are associated with improved outcomes) may be related to unidentified factors that can improve energy preservation thereby preventing cachexia and improving outcomes rather than an inherent advantage in having a higher BMI or elevated serum TG concentrations. In fact, cachexia is a common complication of ESRD and plays a prominent role in the morbidity and mortality associated with this disease given that the risk of death notably increases in patients with ESRD and wasting. (6) In addition, mechanisms that commonly lead to cachexia and PEW are frequently found in patients with ESRD treated with MHD. Therefore, there has been a focus on identifying cachexia-related risk factors which can better explain ESRD-associated mortality, be used to identify patients at the greatest risk of death and provide new potential targets for therapy. (22)

In this regard, data on the association of ECs with clinical and laboratory markers are very limited in ESRD and the only available report is a recent study by Friedman et. al(23), however, given the small sample size and significant gender differences between the control and ESRD group, the findings of this small pilot study were limited. To our knowledge, ours is the first study of serum EC levels in a large cohort of patients with CKD/ESRD who are compared to age- and gender-matched healthy controls. We found significantly increased serum concentrations of 2-AG in patients with CKD/ESRD with the highest serum levels observed in patients undergoing MHD. Serum concentrations of 2-AG positively correlated with serum TG and TG rich lipoprotein levels (VLDL). In addition, serum 2-AG correlated positively with BMI and indices of increased fat mass on body anthropometric measurements. These findings are in line with available literature indicating that higher serum EC levels can be associated with obesity and increased body fat content. (14) Furthermore, serum 2-AG levels negatively correlated with serum HDL-c concentrations, which is consistent with previously published data indicating that CB₁ receptor blockade increases serum HDL-c and ECs down-regulate the expression of apolipoprotein A1, the major protein component of HDL. (19,20,24) We found that the highest tertile of serum 2-AG levels were associated with a significantly decreased risk of death. These associations remained robust after adjustment for age, gender, diabetes, dialysis vintage and inflammation (serum IL-6 levels). In addition, patients with the highest tertile of serum 2-AG had the least number of deaths across different BMI and serum TG strata. Therefore, even in patients with BMI<25 kg/m² or serum TG<126 mg/dL, elevated 2-AG concentrations were associated with lower number of deaths.

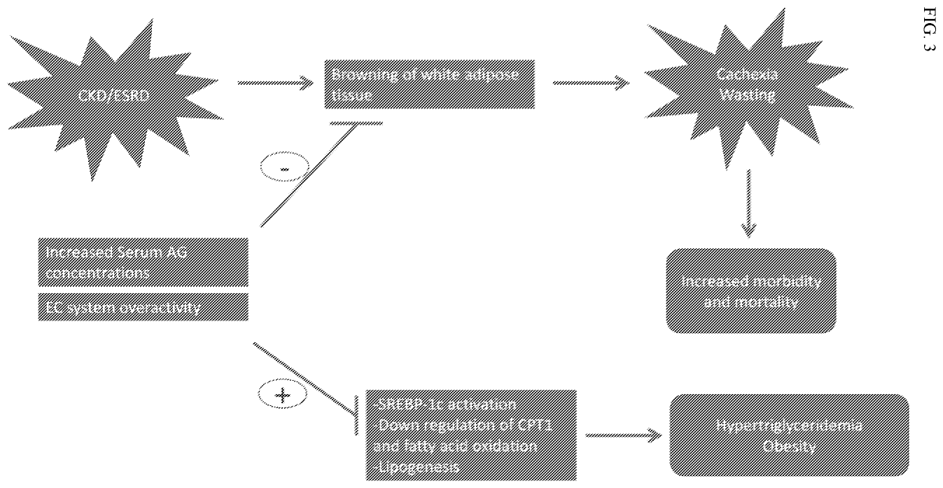

The mechanisms by which increased serum AG levels may play a protective role in ESRD have not been examined. However, recent data on some of the mechanisms involved in the pathogenesis of cachexia in CKD provide important clues to be considered (FIG. 3). It is well known that CKD is associated with wasting of adipose tissue and skeletal muscle through enhanced fat and protein catabolism. Kir et. al. recently described these findings in animals with CKD induced by 5/6 nephrectomy. (10) Weight loss (or lack of weight gain), which is a hallmark of this model, was associated with increased energy expenditure, as shown by elevated O₂ consumption and elevated heat production. Subsequently, they found that the expression of thermogenic genes such as uncoupling protein-1 (UCP-1) was significantly increased in adipose tissue of animals with CKD, an alteration which is termed “browning” of white adipose tissue. The latter processes most likely make a significant contribution to the pathogenesis of CKD-related cachexia and wasting. (9,10) In fact, increased energy expenditure has also been reported in ESRD patients and at least in patients on PD this is associated with increased mortality. (25) Furthermore, common complications of ESRD such as inflammation, hyperparathyroidism and the hemodialysis procedure itself have been associated with increased energy expenditure which increases the risk of cachexia. (26) There is accumulating evidence that the EC system plays a key role in controlling the mechanisms that drive brown adipose tissue thermogenesis. (11, 14, 15) While the role of circulating ECs in this process have not been fully described, it has been shown that reduced levels of 2-AG in forebrain of mice leads to increased brown adipose tissue thermogenesis and energy consumption culminating in a lean phenotype which is resistant to diet-induced obesity. (27) In addition, CB₁ receptor activation in white adipose tissue has been shown to increase the expression of genes associated with adipocyte differentiation, such as peroxisome proliferator-activated receptor-γ (PPARγ) which prevents the transdifferentiation of white adipocytes into the thermogenic brown fat phenotype characterized by increased UCP-1, as observed in the CKD animal model. (28) Therefore, it is possible that increased serum 2-AG levels in ESRD is in response to the CKD-associated browning of white adipose tissue which can increase the risk of cachexia and lead to poor outcomes. In addition, while the possibility that obesity and hypertriglyceridemia directly contribute to elevated serum 2-AG levels cannot be excluded, there is evidence that activation of the EC system via 2-AG may play a causative role in elevated TG levels in ESRD. Dyslipidemia of CKD and ESRD is characterized by increased level of serum TGs and TG rich lipoproteins. (29) One of the proposed mechanisms responsible for these findings is the activation of the nuclear transcription factor sterol regulatory element binding protein-1 c (SREBP-1c) in the liver and adipose tissue of animals with experimental CKD. (30,31) In addition, there is down-regulation of the machinery involved fatty acid (3-oxidation, including decreased peroxisome proliferator activated receptor-α and carnitine palmitoyltransferase-1 (CPT1). Activation of SREBP-1c leads to increased expression of proteins involved in the generation of fatty acids, such as fatty acid synthase, while reduction of CPT1 is associated with reduced fatty acid utilization. Together, these alterations lead to increased TG production and tissue content. It is interesting to note that activation of CB₁ receptors (i.e. via increased 2-AG levels) has been also shown to stimulate SREBP1c and its target enzymes acetyl-CoA carboxylase-1 (ACC1) and fatty acid synthase (FAS) and decrease CPT1 activity and mRNA expression. (32) These effects have also been shown to cause increased serum and hepatic triglyceride content. Therefore, significantly increased serum 2-AG levels, which may indicate overactivity of the EC system in ESRD, might also be partly causing the hypertriglyceridemia observed in this population. Hence, it can be hypothesized that increased serum 2-AG levels may be a compensatory mechanism to counteract ESRD-associated browning of adipose tissue, cachexia and wasting. While the potential amelioration of cachexia in patients with elevated 2-AG levels and EC system overactivity may have protective features, it can also lead to increased risk of obesity and hypertriglyceridemia thereby explaining the association of serum 2-AG levels with BMI and serum TG concentrations (FIG. 3).

While the findings described here are thought-provoking, further mechanistic studies are needed to verify the potential link between serum 2-AG levels and obesity/hypertriglyceridemia paradox. The present study uses non-fasting serum in analysis of lipid-derived mediators. While many of the previous studies of serum EC levels have utilized fasting serum or plasma samples, it should be noted that Cota et. al. have shown that serum levels of 2-AG are not affected by feeding. In addition, we used non-fasting serum across all of the groups in this study including our healthy control. Therefore, introduction of variability based on fasting state of the patients in this study is less likely to play a major role in the associations being reported. The source of 2-AG in the serum needs to be uncover. Some of the potential mechanisms responsible for increased serum 2-AG levels include increased production by the gastrointestinal tract (15), platelet activating factor and its activation in hemodialysis (34,35) and oxidative stress-related modification and inhibition of monoacylglycerol lipase, the main enzyme responsible for 2-AG breakdown. (36)

In conclusion, CKD/ESRD is associated with a significant increase in serum concentrations of the endocannabinoid messenger, 2-AG. ESRD patients on MHD had the highest concentrations of this lipid molecule. In MHD patients, serum concentrations of 2-AG positively correlated with BMI, serum TG concentrations, and clinical markers of body fat content. In addition, higher serum 2-AG concentrations are associated with a significant decrease in risk of death after adjustment for multiple covariates, including inflammation. Patients with the highest tertiles of 2-AG had the least number of deaths regardless of their BMI or serum triglyceride levels.

We sought to examine the association of serum EC levels with clinical parameters and mortality in ESRD patients. Serum concentrations of anandamide and 2-arachidonoyl-sn-glycerol (2-AG) were measured in healthy subjects and patients with advanced chronic kidney disease (CKD) including ESRD on maintenance hemodialysis (MHD). In MHD patients, we examined case-mix-adjusted correlations between serum 2-AG and various clinical/laboratory indices, as well as its association with all-cause mortality. Serum 2-AG levels were significantly increased in CKD patients when compared with controls. MHD patients had the highest 2-AG levels, which positively correlated with body mass index (BMI) (ρ=0.40, p<0.001) and serum triglycerides (TG) (ρ=0.43, p<0.001). Compared to patients with middle tertile of 2-AG, those with the highest tertile had a significantly lower risk of mortality. Furthermore, patients with the highest tertile of serum 2-AG had fewer deaths irrespective of their BMI and TG. In MHD patients, the highest serum 2-AG levels were associated with the lowest risk of death. These findings raise the possibility that overactivity of the EC system as indicated by increased serum 2-AG may be partly responsible for the paradoxical associations observed between hypertriglyceridemia/obesity and reduced mortality in ESRD. Given that ESRD treated with MHD is associated with abnormal energy metabolism, PEW and cachexia, we hypothesized that the serum level of ECs is altered in this patient population. In addition, we sought to determine how alterations in serum EC levels would correlate with laboratory and clinical parameters and ultimately with mortality. We analyzed and compared EC levels in pre-dialysis non-fasting serum samples from patients with ESRD on hemodialysis, healthy controls, patients with stage IV CKD and ESRD on peritoneal dialysis using liquid chromatography/mass spectrometry (LC/MS) techniques.

We have found that serum concentrations of 2-AG are significantly elevated in patients on MHD. Patients with the highest serum concentrations of 2-AG have the best survival and those with the lowest levels (lowest tertile) have the worst survival. Based on this finding and current knowledge of the roles of 2-AG, we postulate that approaches which elevate 2-AG levels in blood to the highest tertile observed in our study patients, will improve survival in patients with end stage renal disease. Specifically, we envisage that the following approaches will be effective: 1) Parenteral (intravenous) administration of a formulation containing appropriate amounts of synthetic 2-AG or one of its lipid precursors (e.g., 1-palmitoyl-2-arachidonoyl-sn-glycerol); 2) Oral or parenteral administration of a compound that increases 2-AG levels by inhibiting the endogenous degradation of 2-AG by monoglycerol lipase (MGL) or other lipase enzymes (e.g. URB602, MGL184); 3) Oral or parenteral administration of a compound that directly activates CB receptors, the molecular target for 2-AG (e.g. cannabis extract containing THC, synthetic THC, synthetic cannabinoid receptor agonists).

### A. STUDY POPULATION

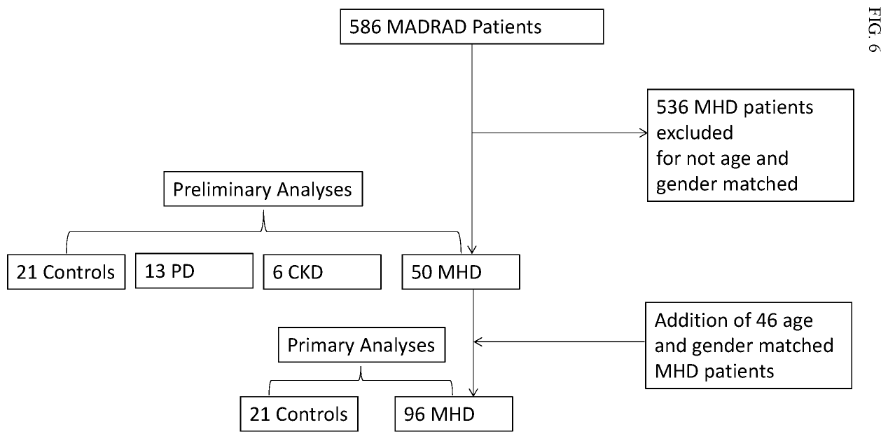

The study population comprised four groups of subjects. The healthy control group (subjects without hypertension, diabetes, other major cardiovascular comorbidities, or medication use) was recruited into this study by the University of California, Irvine (UC Irvine) Institute for Clinical and Translational Science (ICTS). Groups of patients with ESRD on peritoneal dialysis (PD) and non-dialysis CKD stage IV were recruited from the UC Irvine dialysis program and outpatient CKD clinic, respectively. Finally, the MHD group comprised randomly selected subjects from a subcohort of MHD patients enrolled in the initial phase of the Malnutrition, Diet, and Racial Disparities in Chronic Kidney Disease (MADRAD) study (ClinicalTrials.gov #NCT01415570) after being matched to controls on age (±10 years) and gender. MADRAD is a prospective cohort study examining the differences in dietary factors and nutritional status across racial/ethnic groups of MHD patients recruited from outpatient dialysis facilities in the South Bay-Los Angeles, Calif. area. We conducted two phases of analyses. In our preliminary analyses, non-fasting serum levels of AEA and 2-AG in MHD patients (n=50) were compared with age- and gender-matched controls (n=21). Once we identified that 2-AG undergoes the most significant change in ESRD patients, serum was obtained from patients in the CKD and PD groups to further delineate the impact of hemodialysis on 2-AG. Furthermore, to investigate the association of 2-AG with clinical, laboratory, and mortality outcomes, we analyzed 96 age- and gender-matched MHD patients in our primary analyses (FIG. 6). Serum was obtained from MHD patients pre-dialysis during routine weekday hemodialysis treatments, coinciding chronologically with routine blood tests conducted at the outpatient dialysis facilities, and was frozen at −80° C. until analyses were performed.

### B. LIPID EXTRACTION AND ANALYSIS

Serum (0.75 ml) was added methanol (1.5 ml) containing the following internal standards [²H₄]AEA (1 pmol) and [²H₈]2AG (250 pmol). Lipids were extracted using chloroform (3 ml) and 0.1 M sodium chloride (1 ml). The organic phases were dried under N₂, reconstituted in chloroform (2 ml) and applied to open-bed silica gel columns to fractionate lipid groups based on polarity. Eluted fractions containing AEA and AGs (chloroform/methanol, 9:1, v/v) were dried under N₂ and the residue was reconstituted in 60 μL a solvent mixture of chloroform and methanol (1:3, v/v) for LC/MS and LC-MS/MS analyses (for additional details on lipid extraction and analysis, see supplementary material).

Anandamide analysis by LC/MS. Anandamide levels were measured using an LC system consisting of an Agilent 1100 system and 1946D mass spectrometer detector equipped with electrospray ionization interface (Agilent Technologies, Santa Clara, Calif., USA) (37). The fatty acid ethanolamides include AEA were separated on a ZORBAX Eclipse XDB-C18 column (2.1×100 mm, 1.8 μm, Agilent Technologies) using an acetonitrile gradient. Solvent A consisted of water containing 0.1% formic acid, and Solvent B consisted of acetonitrile containing 0.1% formic acid. The gradient profile of the solvents was as follows: 0-15 min, 65% B; 15-16 min, 65-100% B linear gradient; 16-26 min, 100% B; 26-28 min, 100-65% B linear gradient; 28-30 min, 65% B. The flow rate was 0.3 ml/min and the column temperature was maintained at 15° C. Electrospray ionization interface was in the positive ionization mode, capillary voltage was set at 3 kV, and the fragment or voltage was set at 70 V. N₂ was used as a drying gas at a flow rate of 12 liters/min and a temperature of 350° C. The nebulizer pressure was set at 40 psi. Selected ion monitoring (SIM) mode was used to monitor protonated molecular ions [M+H]⁺ of AEA and [²H₄]AEA. Absolute amounts of AEA was quantified using a calibration curve.

AG analysis by LC/MS/MS. AG levels were measured using an LC system consisting of an Agilent 1200 system and 6410 Triple Quadrupole mass spectrometer detector equipped with electrospray ionization interface (Agilent Technologies, Santa Clara, Calif., USA). AGs were separated on a ZORBAX Eclipse XDB-C18 column (2.1×100 mm, 1.8 μm, Agilent Technologies) using a methanol gradient. Solvent A consisted of water containing 5 mM ammonium acetate and 0.25% acetic acid, and Solvent B consisted of methanol containing 5 mM ammonium acetate and 0.25% acetic acid. The gradient profile of the solvents was as follows: 0-7 min, 100% B; 7-8 min, 100-90% B linear gradient, 8-10 min, 90% B. The flow rate was 1 ml/min, and the column temperature was maintained at 40° C. Electrospray ionization interface was in the positive ionization mode, capillary voltage was set at 4 kV, with a delta EMV of 0.4 kV. N₂ was used as a drying gas at a flow rate of 12 liters/min and a temperature of 350° C. and the nebulizer pressure was set at 50 psi. Fragment voltage and collision energy were 135 eV and 10 eV for both AG and d⁸-2AG. Multiple reaction monitoring (MRM) was used to quantify AG and d⁸-2AG, as internal standard: m/z 379→287 for AG, 387→295 for d⁸-2AG. Absolute amounts of AGs were quantified using a calibration curve.

### C. EXPOSURE AND OUTCOME ASSESSMENT

Our primary exposure was serum 2-AG categorized into tertiles (<55.97, 55.97-<117.16, and ≥117.16 pmol/ml) among 96 MHD patients. The main outcome was all-cause mortality. Follow-up started at the date of serum 2-AG measurement until death, transplantation, loss-to-follow-up, or end of study period. Data on all censoring events were obtained by MADRAD study coordinators every six months and were reviewed by MADRAD study nephrologists.

### D. STATISTICAL ANALYSIS

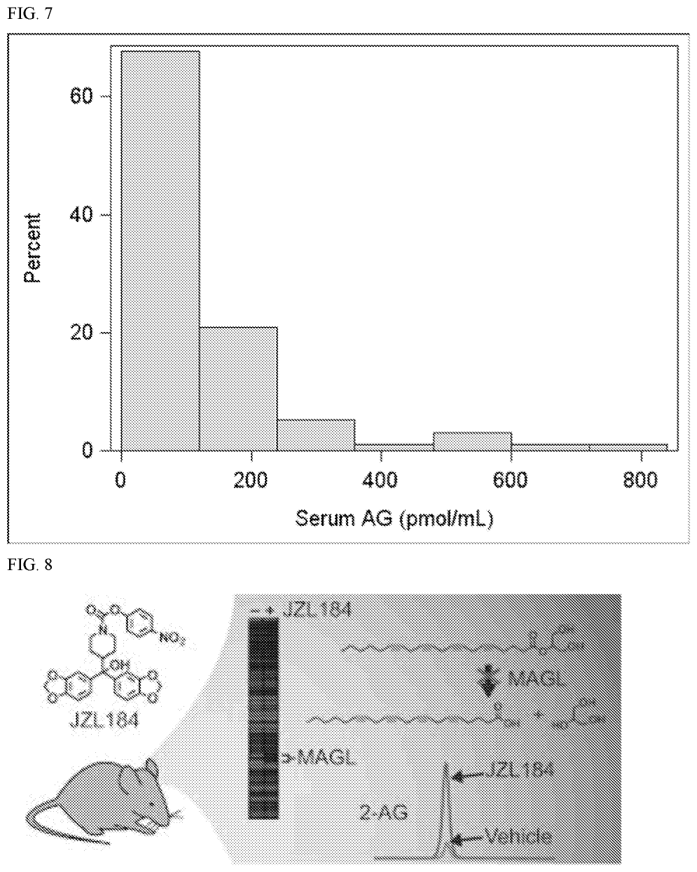

Data were summarized using means (±standard deviation, SD), median (interquartile range, IQR) or proportions, where appropriate. Comparisons between controls, CKD, and ESRD patients were performed with Wilcoxon-Mann-Whitney U or Kruskal-Wallis tests, where appropriate. We also conducted ANCOVA analyses across control, CKD, MHD, and PD groups adjusting for age, gender, race, ethnicity, and diabetes status. Characteristics of MHD patients across 2-AG tertiles were compared using trend tests. Serum 2-AG was tested for normality with formal and visual tests (FIG. 7). We analyzed the association of serum 2-AG with all-cause mortality using Cox proportional hazards models, under the following models: (i) Model 1: Unadjusted; (ii) Model 2: Adjusted for case-mix variables (age, gender, race, and ethnicity); (iii) Model 3: Adjusted for covariates in Model 2, plus diabetes and dialysis vintage; and (iv) Model 4: Adjusted for covariates in Model 3, plus serum IL-6. Furthermore, we examined the number of deaths stratified by dichotomized groups of BMI and serum TG, with cutoffs dictated by the cohort distribution and/or clinical relevance. Finally, we calculated unadjusted and adjusted (Model 3) Spearman correlation coefficients to describe the relationship between 2-AG and clinical and laboratory markers. Data on BMI were primarily sourced from LDO electronic records, or imputed with available BMI levels collected by MADRAD study coordinators for those missing BMI (14%). Missing data on serum IL-6 (<0.05%) were imputed by the mean of the cohort. Two-sided p-values<0.05 were considered significant. Analyses were performed using SAS, version 9.4 (SAS Institute Inc, Cary, N.C.).

### E. EC LEVELS

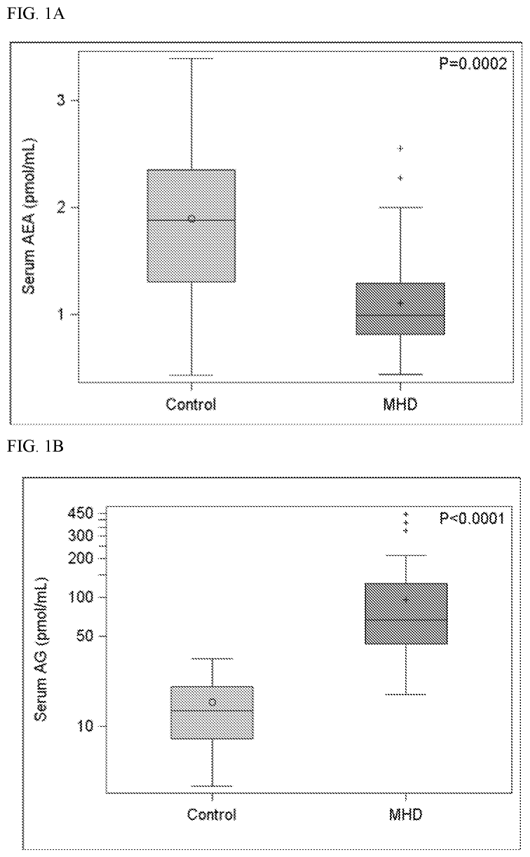

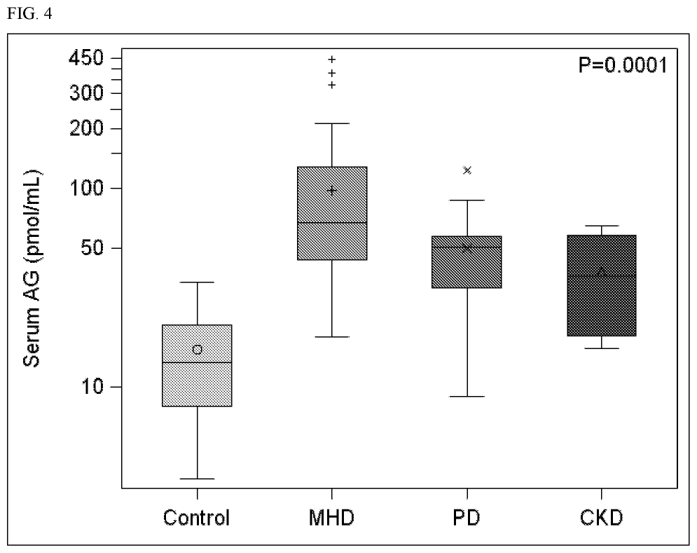

We first examined serum EC levels in controls compared to MHD patients (n=50). Subsequently, we compared 2-AG in controls, CKD, PD, and MHD patients. Among the 4 groups, MHD patients had a larger proportion of Hispanics and females Table 6 (Supplement Table 1). We did not find differences in serum AEA levels across strata of demographic characteristics Table 7 (Supplement Table 2). An initial assessment of AEA and serum 2-AG showed that there was a difference in EC levels in controls and 50 MHD patients (FIG. 1A-1B). We found that serum AEA concentrations were lower in MHD patients versus controls (mean±SD, 1.11±0.44 and 1.89±0.76 pmol/ml, respectively; FIG. 1A). In contrast, serum 2-AG was several folds elevated in MHD patients versus controls (median (IQR), 67 (44-128) and 13 (8-20) pmol/ml, respectively; FIG. 1B). In view of the higher 2-AG levels in MHD patients, we sought to determine whether CKD has an impact on serum 2-AG and the extent to which dialysis modality alters serum 2-AG concentrations. While MHD patients had the highest 2-AG levels, we found that patients in the PD and CKD groups also had elevated 2-AG (median (IQR), 50 (32-57) and 36 (18-58) pmol/ml, for PD and CKD patients respectively, FIG. 4) versus controls. The observed differences in 2-AG across controls, MHD, PD and CKD patients persisted after demographic adjustment (e.g., age, gender, race, ethnicity and the presence of diabetes (P<0.0001)).

### F. COHORT CHARACTERISTICS

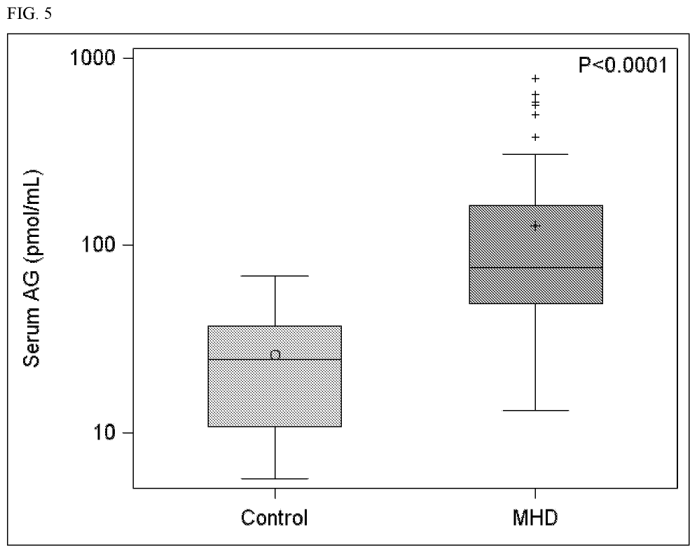

Baseline characteristics of the 96 MHD patients are presented in Table 1. The cohort was (mean±SD) 52±12 years old with 64% females and 52% diabetics. The median (IQR) serum 2-AG was 76 (49-163) pmol/ml, and differed from controls (FIG. 5). Patients with higher 2-AG were more likely to have higher BMI, TG, non-HDL (high-density lipoprotein) and very low density lipoprotein (VLDL) cholesterol. There were no differences across demographics in the 96 MHD patients Table 7 (Supplement Table 2).

Demographics, Clinical and Laboratory Characteristics for MHD Patients. Baseline demographic and clinical data, including age, gender, race, and ethnicity, were obtained by the MADRAD study coordinators. Diabetes as a pre-existing comorbid condition was ascertained by MADRAD study coordinators and study dietitians according to patient self-reported history and obtained via ICD-9 codes at the time of study entry. Dialysis vintage for MHD patients was calculated as the interval of time between the date of the patient's first dialysis treatment and the date of serum AG measurement.

Routine laboratory measurements, including lipid panels were obtained from the dialysis facilities' electronic records. Blood samples were drawn using standardized techniques and measured using automated and standardized methods at a central laboratory in Deland, Fla., typically within 24 hours. An extended serum lipid panel was measured at the UC Irvine Medical Center laboratory. Very low density lipoprotein concentrations were measured and not calculated. Serum concentrations of interleukin (IL)-6 were determined using ELISA assay kits from R&D systems (Minneapolis, Minn.) and Affymetrix ThermoFisher Scientific per manufacturer's protocol.

Data on body mass index (using post-dialysis weight) were also obtained from electronic records of the LDO. In addition, patient body composition surrogates were measured by MADRAD study coordinators during treatment visits. Further details about the MADRAD study ascertainment of body anthropometry have been previously reported (38).

To assess depression and the severity of its symptoms over the past two weeks, patients completed the Beck Depression Inventory-II (BDI) questionnaire. The BDI score is the sum of the responses to 21 questions each ranked on a scale from 0-3. Patients also completed the Short Form 36 (SF36) quality of life questionnaire. The individual question responses were scored and then calculated to assess patient physical and mental health domains, as well as eight dimensions of health: physical functioning, role limitations due to physical health, role limitations due to personal or emotional problems, energy/fatigue, emotional well-being, social functioning, bodily pain and general health (39).

For all laboratory and health questionnaire measurements, the closest measurement, or questionnaire score prior to the AG date of measurement were used in analyses.

TABLE 1
Baseline Characteristics of 96 Maintenance Hemodialysis Patients According to Serum 2-AG Tertiles.

| | | Serum 2-AG (pmol/mL) | | | |
|---|---|---|---|---|---|
| Variable | Total | <55.97 | 55.97-<117.16 | ≥117.16 | p-value |
| N (%) | 96 | 32 (33) | 32 (33) | 32 (33) | |
| Age (years) | 52 ± 12 | 54 ± 13 | 52 ± 9 | 50 ± 12 | 0.17 |
| Female (%) | 64 | 63 | 56 | 72 | 0.44 |
| Race (%) | | | | | |
| White | 83 | 78 | 81 | 91 | 0.18 |
| Asian | 17 | 22 | 19 | 9 | 0.18 |
| Ethnicity (%) | | | | | |
| Hispanic | 53 | 47 | 50 | 63 | 0.21 |
| Diabetes (%) | 52 | 53 | 50 | 53 | 1 |
| Body mass index (kg/m²) | 27.9 ± 6.3 | 24.1 ± 4.4 | 29.1 ± 6.8 | 30.5 ± 5.8 | <0.0001 |
| Laboratory tests | | | | | |
| Albumin (g/dL) | 4.0 ± 0.3 | 3.9 ± 0.3 | 4.0 ± 0.3 | 4.0 ± 0.3 | 0.6 |
| Creatinine (mg/dL) | 9.3 ± 3.1 | 8.9 ± 3.1 | 9.5 ± 3.0 | 9.5 ± 3.2 | 0.48 |
| Ferritin (ng/mL) | 619 (387, 886) | 618 (338, 804) | 604 (407, 829) | 628 (366, 937) | 0.57 |
| TIBC (mg/dL) | 231.2 ± 38.2 | 215.1 ± 31.7 | 237.4 ± 37.0 | 237.9 ± 41.0 | 0.03 |
| PTH (pg/mL) | 380 (265, 576) | 447 (231, 651) | 365 (265, 609) | 380 (301, 516) | 0.89 |
| Lipid panel | | | | | |
| VLDL (mg/dL) | 12 (6, 28) | 9 (6, 13) | 11 (6, 22) | 29 (13, 46) | <0.0001 |
| Triglycerides (mg/dL) | 126 (92, 213) | 104 (73, 134) | 120 (92, 166) | 221 (127, 287) | <0.0001 |
| Cholesterol (mg/dL) | 143.1 ± 38.8 | 135.5 ± 38.7 | 140.9 ± 41.0 | 152.8 ± 35.8 | 0.07 |
| HDL Cholesterol (mg/dL) | 42.2 ± 20.2 | 47.9 ± 22.1 | 42.7 ± 20.5 | 36.2 ± 16.5 | 0.02 |
| LDL Cholesterol (mg/dL) | 77.3 ± 28.4 | 73.7 ± 32.5 | 76.4 ± 27.7 | 81.7 ± 25.0 | 0.26 |
| LPA (mg/dL) | 2 (1, 4) | 2 (1, 4) | 2 (1, 4) | 3 (1, 5) | 0.25 |
| NHDL (mg/dL) | 100.8 ± 39.2 | 87.6 ± 35.3 | 98.2 ± 42.6 | 116.7 ± 34.7 | 0.003 |
| IL-6 (pg/mL) | 2 (1, 5) | 3 (1, 5) | 2 (1, 5) | 2 (1, 4) | 0.45 |
| Vintage (%) | | | | | 0.74 |
| <366 days | 16 | 9 | 25 | 13 | |
| 366-<1095 days | 27 | 25 | 22 | 34 | |
| ≥1095 days | 57 | 66 | 53 | 53 | |

Note:
Data are presented as percentages, mean ± standard deviation or median (interquartile range), where appropriate. P-values were calculated by parametric and non-parametric tests for trend, where applicable. Abbreviations: TIBC, total iron-binding capacity; PTH, parathyroid hormone; VLDL, very low-density lipoprotein; HDL, high-density lipoprotein; LDL, low-density lipoprotein; LPA, lipoprotein(a); NHDL, non-high-density lipoprotein; IL-6, Interleukin-6.

### G. CORRELATION OF SERUM 2-AG WITH CLINICAL AND LABORATORY INDICES

Serum 2-AG positively correlated with BMI, mid-arm muscle circumference, biceps and triceps skin fold, serum TG and VLDL after Model 3 adjustment (Table 2). However, serum 2-AG negatively correlated with serum HDL cholesterol (HDL-c) (ρ=−0.33). Correlation coefficients of 2-AG with other clinical and laboratory data are presented in Table 8 (Supplement Table 3).

TABLE 2
Unadjusted and Model 3-Adjusted Spearman Correlation Coefficients
of Serum 2-AG and Relevant Laboratory, Body Anthropometric,
Quality of Life and Depression Data.

| | Unadjusted | | Model 3-Adjusted | |
|---|---|---|---|---|
| Variable | ρ | p-value | ρ | p-value |
| Laboratory Tests | | | | |
| Albumin (g/dL) | 0.05 | 0.62 | 0.08 | 0.47 |
| Creatinine (mg/dL) | 0.02 | 0.86 | 0.03 | 0.83 |
| Ferritin (ng/mL) | 0.03 | 0.78 | 0.07 | 0.56 |
| TIBC (mcg/dL) | 0.28 | 0.01 | 0.32 | 0.004 |
| PTH (pg/mL) | −0.02 | 0.87 | −0.002 | 0.98 |
| Lipid Panel | | | | |
| VLDL (mg/dL) | 0.44 | <0.0001 | 0.42 | <0.0001 |
| Triglycerides (mg/dL) | 0.47 | <0.0001 | 0.43 | <0.0001 |
| Cholesterol (mg/dL) | 0.28 | 0.005 | 0.23 | 0.03 |
| HDL Cholesterol (mg/dL) | −0.31 | 0.002 | −0.33 | 0.001 |
| LDL Cholesterol (mg/dL) | 0.18 | 0.09 | 0.13 | 0.21 |
| LPA (mg/dL) | 0.2 | 0.05 | 0.21 | 0.05 |
| NHDL (mg/dL) | 0.37 | 0.0003 | 0.33 | 0.001 |
| IL-6 (pg/mL) | −0.05 | 0.65 | 0.009 | 0.94 |
| Body mass index (kg/m²) | 0.43 | <0.0001 | 0.4 | <0.0001 |
| Body Anthropometry | | | | |
| Biceps skin fold (mm) | 0.34 | 0.0008 | 0.32 | 0.002 |
| Triceps skin fold (mm) | 0.33 | 0.001 | 0.32 | 0.002 |
| Mid-arm muscle circ. (mm) | 0.34 | 0.0009 | 0.33 | 0.002 |
| Mid-arm circ. (mm) | 0.09 | 0.4 | 0.12 | 0.29 |
| NIR body fat % | 0.31 | 0.002 | 0.31 | 0.004 |
| Quality of Life | | | | |
| Physical functioning | 0.03 | 0.82 | 0.07 | 0.52 |
| Role limitations due to physical health | 0.18 | 0.11 | 0.22 | 0.05 |
| Role limitations due to emotional problems | 0.08 | 0.47 | 0.08 | 0.51 |
| Energy/fatigue | 0.03 | 0.78 | 0.05 | 0.65 |
| Emotional well-being | −0.03 | 0.76 | 0.02 | 0.86 |
| Social functioning | 0.04 | 0.71 | 0.07 | 0.54 |
| Pain | 0.02 | 0.86 | 0.05 | 0.67 |
| General health | 0.1 | 0.37 | 0.16 | 0.16 |
| Physical health | 0.11 | 0.31 | 0.17 | 0.14 |
| Mental health | 0.06 | 0.61 | 0.09 | 0.44 |
| Beck Depression Index | | | | |
| BDI Score | −0.04 | 0.73 | −0.05 | 0.66 |

Abbreviations:
TIBC, total iron-binding capacity;
PTH, parathyroid hormone;
VLDL, very low-density lipoprotein;
HDL, high-density lipoprotein;
LDL, low-density lipoprotein;
LPA, lipoprotein(a);
NHDL, non-high-density lipoprotein;
IL-6, Interleukin-6;
circ., circumference;
NIR, near-infrared

TABLE 3
Association of serum AG and all-cause mortality in 96 MHD patients with 4-level adjustment

| | | Model 1 | | Model 2 | | Model 3 | | Model 4 | |
|---|---|---|---|---|---|---|---|---|---|
| | | (n = 96) | | (n = 96) | | (n = 96) | | (n = 96) | |
| Serum | No. of | HR | | HR | | HR | | HR | |
| AG | deaths | (95% | p- | (95% | p- | (95% | p- | (95% | p- |
| (pmol/mL) | (col. %) | CI) | value | CI) | value | CI) | value | CI) | value |
| <55.97 | 9 | 1.52 | 0.43 | 0.62 | 0.43 | 0.62 | 0.45 | 0.70 | 0.59 |
| | (56%) | (0.54-4.28) | | (0.19-2.02) | | (0.18-2.18) | | (0.19-2.60) | |
| 55.97 to | 6 | Reference | | Reference | | Reference | | Reference | |
| <117.16 | (38%) | | | | | | | | |
| 117.16 or | 1 | 0.12 | 0.05 | 0.06 | 0.01 | 0.05 | 0.01 | 0.05 | 0.02 |
| more | (6%) | (0.02-1.04) | | (0.01-0.57) | | (0.01-0.56) | | (0.004-0.63) | |

Model 1: Unadjusted; Model 2: Adjusted for case-mix variables, which included age, gender, race, and ethnicity; Model 3: Adjusted for covariates in Model 2, plus diabetes and dialysis vintage; Model 4: Adjusted for covariates in Model 3, plus inflammation (serum IL-6).

TABLE 4
No. of death events in AG tertiles across BMI strata

| A | BMI (kg/m2) | | | |
|---|---|---|---|---|
| Serum AG | <25 | | ≥25 | |
| (pmol/mL) | No. of patients (%) | No. of deaths (%) | No. of patients (%) | No. of deaths (%) |
| <55.97 | 17 (55) | 4 (67) | 15 (23) | 5 (50) |
| 55.97 to <117.16 | 9 (29) | 2 (33) | 23 (35) | 4 (40) |
| ≥117.16 | 5 (16) | 0 (0) | 27 (42) | 1 (10) |
| Total | 31 | 6 | 65 | 10 |
| B | BMI (kg/m2) | | | |
| Serum AG | <27 | | ≥27 | |
| (pmol/mL) | No. of patients (%) | No. of deaths (%) | No. of patients (%) | No. of deaths (%) |
| <55.97 | 22 (46) | 5 (63) | 10 (21) | 4 (50) |
| 55.97 to <117.16 | 16 (33) | 3 (38) | 16 (33) | 3 (38) |
| ≥117.16 | 10 (21) | 0 (0) | 22 (46) | 1 (13) |
| Total | 48 | 8 | 48 | 8 |
| C | BMI (kg/m2) | | | |
| Serum AG | <30 | | ≥30 | |
| (pmol/mL) | No. of patients (%) | No. of deaths (%) | No. of patients (%) | No. of deaths (%) |
| <55.97 | 29 (45) | 8 (62) | 3 (10) | 1 (33) |
| 55.97 to <117.16 | 20 (31) | 4 (31) | 12 (39) | 2 (67) |
| ≥117.16 | 16 (25) | 1 (8) | 16 (52) | 0 (0) |
| Total | 65 | 13 | 31 | 3 |

TABLE 5
No. of death events in AG tertiles across TG strata

| A | Triglycerides (mg/dL) | | | |
|---|---|---|---|---|
| Serum AG | <126 | | ≥126 | |
| (pmol/mL) | No. of patients (%) | No. of deaths (%) | No. of patients (%) | No. of deaths (%) |
| <55.97 | 22 (46) | 7 (50) | 10 (21) | 2 (100) |
| 55.97 to <117.16 | 18 (38) | 6 (43) | 14 (29) | 0 (0) |
| ≥117.16 | 8 (17) | 1 (7) | 24 (50) | 0 (0) |
| Total | 48 | 14 | 48 | 2 |
| B | Triglycerides (mg/dL) | | | |
| Serum AG | <160 | | ≥160 | |
| (pmol/mL) | No. of patients (%) | No. of deaths (%) | No. of patients (%) | No. of deaths (%) |
| <55.97 | 27 (45) | 7 (50) | 5 (14) | 2 (100) |
| 55.97 to <117.16 | 23 (38) | 6 (43) | 9 (25) | 0 (0) |
| ≥117.16 | 10 (17) | 1 (7) | 22 (61) | 0 (0) |
| Total | 60 | 14 | 36 | 2 |

TABLE 6
(Supplement TABLE 1). Baseline Characteristics According
to 21 Control Subjects, 6 CKD, 13 PD and 50 MHD patients

| | Group | | | |
|---|---|---|---|---|
| Variable | Control | CKD | PD | HD |
| N | 21 | 6 | 13 | 50 |
| Age (years) | 49 ± 9 | 72 ± 12 | 48 ± 14 | 52 ± 11 |
| Female (%) | 67 | 17 | 62 | 68 |
| Race (%) | | | | |
| White | 86 | 100 | 46 | 82 |
| Asian | 14 | 0 | 54 | 18 |
| Hispanic ethnicity (%) | 38 | 0 | 31 | 52 |
| Diabetes (%) | 0 | 33 | 38 | 52 |

TABLE 7
(Supplement TABLE 2). Serum AEA levels in 50 MHD
patients and Serum AG levels in 96 MHD patients
stratified by demographic characteristics.

| | Endocannabinoids | | | |
|---|---|---|---|---|
| | Serum AEA | | Serum AG | |
| | (n = 50) | | (n = 96) | |
| Subgroup | Mean ± SD | p-value | Median (IQR) | p-value |
| Age (years) | | | | |
| <50 | 1.02 ± 0.36 | 0.27 | 73 (51, 161) | 0.73 |
| ≥50 | 1.16 ± 0.48 | | 78 (48, 165) | |
| Gender | | | | |
| Female | 1.06 ± 0.43 | 0.29 | 81 (53, 177) | 0.25 |
| Male | 1.20 ± 0.45 | | 75 (44, 127) | |
| Race | | | | |
| White | 1.13 ± 0.45 | 0.35 | 80 (48, 172) | 0.19 |
| Asian | 0.98 ± 0.35 | | 67 (51, 101) | |
| Ethnicity | | | | |
| Hispanic | 1.08 ± 0.36 | 0.63 | 83 (52, 216) | 0.13 |
| Non-Hispanic | 1.14 ± 0.51 | | 73 (48, 127) | |
| Dialysis vintage (days) | | | | |
| <1095 | 1.07 ± 0.44 | 0.61 | 96 (51, 172) | 0.42 |
| ≥1095 | 1.13 ± 0.44 | | 72 (48, 144) | |
| Presence of diabetes | | | | |
| Yes | 1.17 ± 0.44 | 0.28 | 78 (48, 174) | 0.63 |
| No | 1.04 ± 0.42 | | 75 (49, 142) | |

TABLE 8
(Supplement TABLE 3). Correlations for all lab data

| | Unadjusted | | Model 3-Adjusted | |
|---|---|---|---|---|
| Variable | ρ | p-value | ρ | p-value |
| Age (years) | −0.12 | 0.28 | 0.05 | 0.67 |
| Alkaline Phosphatase (IU/L) | 0.1 | 0.39 | 0.1 | 0.4 |
| Basophils (%) | 0.09 | 0.41 | 0.07 | 0.58 |
| Bicarbonate (meq/L) | −0.16 | 0.15 | −0.11 | 0.33 |
| BSA (DuBois) (m2) | 0.31 | 0.004 | 0.4 | 0.0003 |
| BUN (mg/dL) | 0.1 | 0.39 | 0.07 | 0.54 |
| Calcium (mg/dL) | −0.09 | 0.4 | −0.08 | 0.47 |
| Calcium corrected (mg/dL) | −0.09 | 0.39 | −0.09 | 0.42 |
| Ca × phos corrected | −0.02 | 0.86 | −0.05 | 0.65 |
| Chloride (meq/L) | 0.06 | 0.62 | 0.1 | 0.4 |
| Dialyzer flow Qd (mL/min) | −0.08 | 0.46 | −0.12 | 0.3 |
| Dialyzer KoA | −0.06 | 0.56 | −0.09 | 0.44 |
| eKdt/V dialysis | −0.13 | 0.22 | −0.17 | 0.15 |
| Eosinophils (%) | −0.09 | 0.4 | −0.07 | 0.55 |
| Globulin (g/dL) | 0.23 | 0.04 | 0.22 | 0.06 |
| Height (inches) | 0.04 | 0.75 | 0.14 | 0.21 |
| Hemoglobin (g/dL) | 0.24 | 0.03 | 0.23 | 0.04 |
| Hours/week treated (hours) | 0.05 | 0.63 | 0.13 | 0.27 |
| Iron (μg/dL) | 0.08 | 0.49 | 0.06 | 0.61 |
| Iron saturation (%) | −0.04 | 0.71 | −0.07 | 0.56 |
| Kt/V prescribed | −0.17 | 0.13 | −0.24 | 0.04 |
| LDH total (U/L) | 0.03 | 0.81 | −0.04 | 0.75 |
| Lymphocytes (%) | −0.02 | 0.86 | −0.04 | 0.72 |
| MCH (pg) | 0.14 | 0.21 | 0.13 | 0.26 |
| MCHC (g/dL) | 0.23 | 0.04 | 0.24 | 0.03 |
| MCV (fL) | −0.09 | 0.44 | −0.1 | 0.37 |
| Minutes dialyzed (min.) | 0.1 | 0.35 | 0.16 | 0.16 |
| Monocytes (%) | −0.11 | 0.34 | −0.06 | 0.63 |
| MPV (fL) | −0.01 | 0.96 | −0.03 | 0.79 |
| Neutrophils (%) | 0.11 | 0.34 | 0.11 | 0.36 |
| No. of days/week treated | 0.17 | 0.11 | 0.18 | 0.13 |
| nPCR (g/kg/d) | −0.12 | 0.29 | −0.13 | 0.25 |
| Phosphorus (mg/dL) | −0.04 | 0.73 | −0.07 | 0.55 |
| Platelet count (mm3) | 0.08 | 0.48 | 0.06 | 0.66 |
| Potassium (meq/L) | −0.07 | 0.54 | −0.12 | 0.31 |
| Protein total (g/dL) | 0.19 | 0.09 | 0.21 | 0.07 |
| Red blood cell (mm6) | 0.1 | 0.38 | 0.11 | 0.35 |
| RDW (%) | −0.17 | 0.13 | −0.2 | 0.08 |
| SGOT (AST) (U/L) | 0.02 | 0.85 | 0.03 | 0.77 |
| SGPT (ALT) (U/L) | 0.11 | 0.38 | 0.16 | 0.2 |
| Sodium (meq/L) | 0.003 | 0.98 | 0.06 | 0.58 |
| spKdt/V dialysis | −0.14 | 0.19 | −0.19 | 0.1 |
| spKt/V total | −0.12 | 0.28 | −0.15 | 0.18 |
| TBW (Watson) (L) | 0.25 | 0.02 | 0.43 | <0.0001 |
| UIBC (μg/dL) | 0.22 | 0.04 | 0.27 | 0.02 |
| URR (%) | −0.12 | 0.29 | −0.16 | 0.17 |
| White blood cell (×1000 mm3) | 0.05 | 0.64 | 0.05 | 0.68 |
| Weight (kg) | 0.4 | 0.0001 | 0.45 | <0.0001 |
| Post-dialysis weight (kg) | 0.38 | 0.0004 | 0.42 | 0.0001 |
| Pre-dialysis weight (kg) | 0.39 | 0.0003 | 0.43 | <0.0001 |

Footnote:
BSA (DuBois), body surface area; BUN, blood urea nitrogen; ca × phos corrected, calcium × phosphorous corrected; dialyzer flow Qd, dialyzer flow rate; eKdt/V, estimated Kdt/V; LDH, lactic acid dehydrogenase; MCH, mean corpuscular hemoglobin; MCHC, mean corpuscular hemoglobin concentration; MCV, mean corpuscular volume; MPV, mean platelet volume; nPCR, normalized protein catabolic rate; RDW, red blood cell distribution width; SGOT (AST), serum glutamic oxaloacetic transaminase (aspartate aminotransferase); SGOT (ALT), alanine aminotransferase; spKt/V, single pool KTV; TBW (Watson), total body water (Watson formula); UIBC, unsaturated iron binding capacity; URR, urea reduction ratio.

### H. ASSOCIATION OF SERUM 2-AG AND MORTALITY

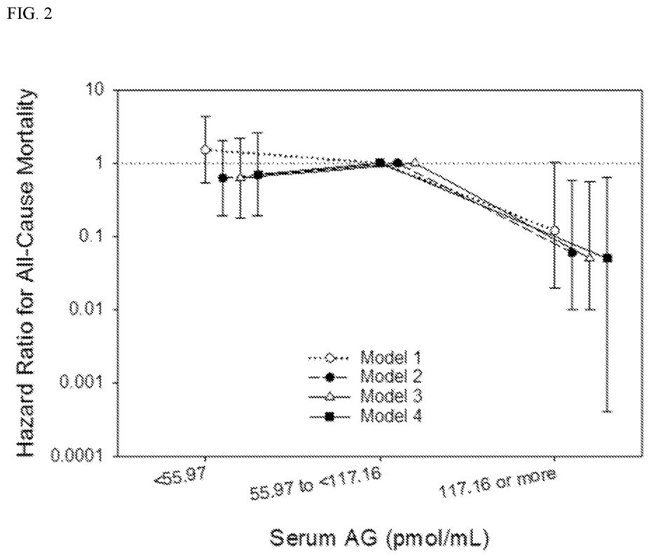

Among 96 patients, we observed a crude death rate of 9.1 [95% CI: 4.6-13.5] per 100 person-years. The highest 2-AG tertile was associated with lower mortality compared to the middle tertile across all adjustment levels (FIG. 2, Table 3). Additionally, MHD patients in the highest 2-AG tertile had the lowest number of deaths across BMI cutoffs (Tables 4 and 5). Likewise, patients in the highest 2-AG tertile had the lowest number of deaths irrespective of TG cutoffs (Tables 4 and 5).

### I. STUDY OF MGL INHIBITOR IN ANIMAL MODEL OF CHRONIC KIDNEY DISEASE

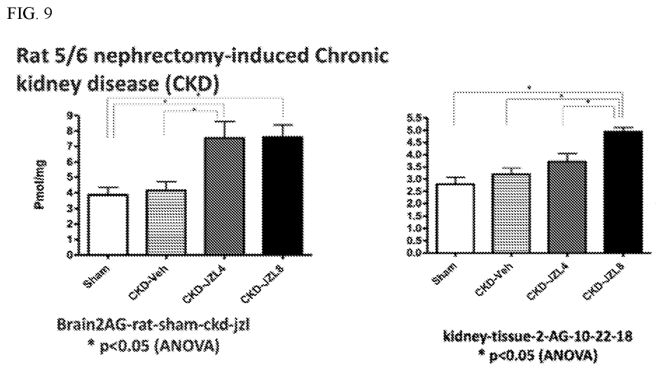

We used a well-known experimental tool, JZL184, which inhibits the enzyme responsible for breakdown of 2-AG, monoacylglycerol lipase (MGL), to determine the effect of increased tissue 2-AG levels on markers of renal function in animal models of chronic kidney disease (CKD). JZL184 was administered intraperitoneally in a well-established rat and mouse model of chronic kidney disease (CKD) (FIG. 8).

Using this experimental tool (JZL184) has been shown to significantly increase brain and kidney levels of 2-AG. Therefore, we induced CKD in animals via 5/6 nephrectomy (surgical removal of one entire kidney in addition to 2/3 of the contralateral kidney which results in significant renal mass reduction and development of kidney failure over 6-10 weeks) in two separate studies (one set in rats and one set in mice). We subsequently treated the animals with doses of JZL184 which have been shown to increase 2-AG levels without causing overt symptoms of endocannabinoid activation in the central nervous system (4 mg/kg in mice and 4-8 mg/kg in rats) for a total of 4 weeks.

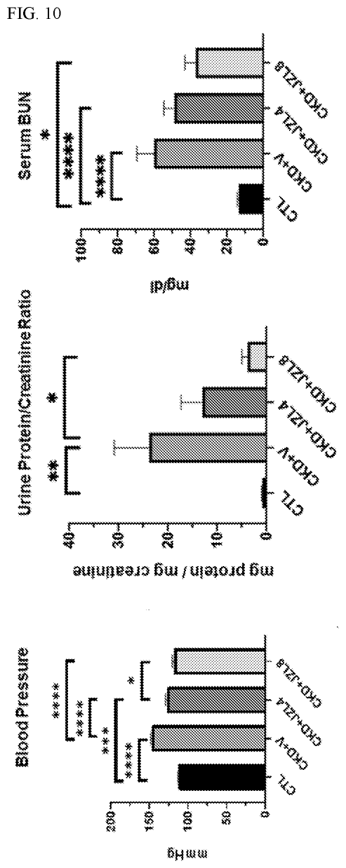

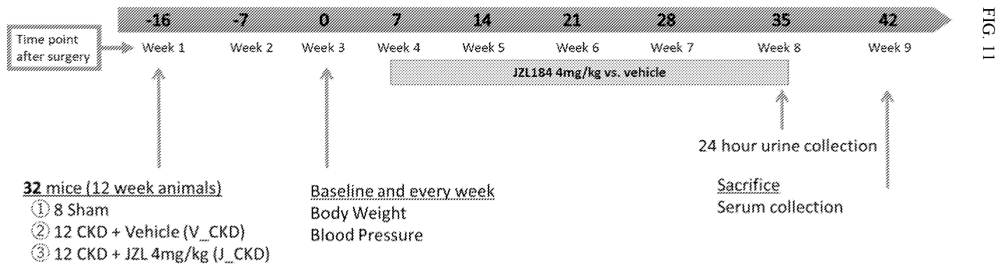

In the rats, male Sprague-Dawley rats underwent 5/6 nephrectomy versus sham surgery and after two weeks were randomized to 4 groups: sham surgery, CKD treated with vehicle, CKD treated with 4 mg/kg JZL184 and CKD treated with 8 mg/kg JZL184 (n=6-8). During and at the end of the study, non-invasive blood pressure measurements were performed using tail-cuff plethysmography. Before sacrifice, a 24-hour urine sample was collected and evaluated for urine protein and creatinine to assess for degree of proteinuria (degree of proteinuria is a marker of glomerular and interstitial kidney damage in advanced CKD). After sacrifice, serum was obtained to assess for renal function. We found that treatment with JZL184 resulted in a significant increase in renal and cerebral cortex 2-AG concentration (FIG. 9). This was associated reduced systolic blood pressure and decreased rate of urine protein excretion. There was also a signal toward improved renal function (FIG. 10).

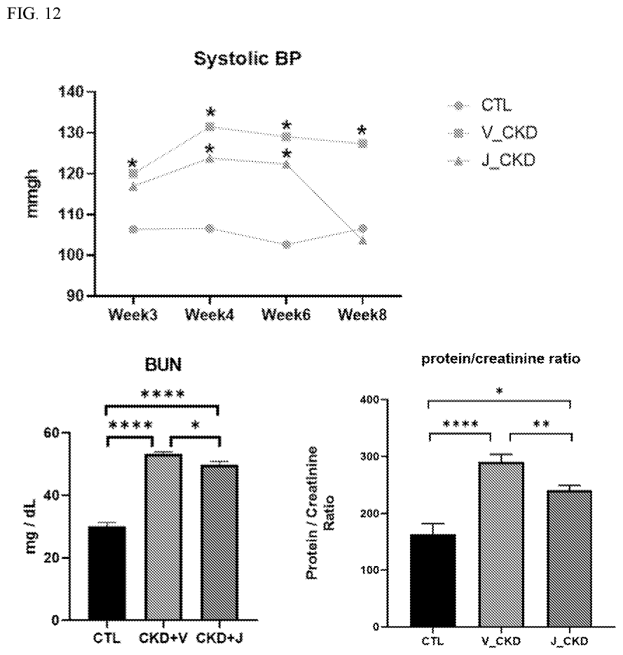

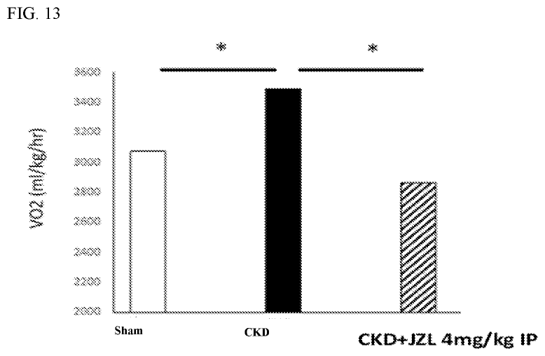

We performed a similar experiment in mice. Male C57BL/6J mice underwent sham surgery versus 5/6 nephrectomy to induce CKD and two weeks after surgery were randomized to vehicle versus JZL184 therapy (4 mg/kg). Treated animals received JZL184 (4 mg/kg) for an additional 4 weeks (FIG. 11). We again noted that treatment with JZL184 was associated with a significant improvement in blood pressure, and reduced serum BUN concentration and decreased urinary protein excretion and (FIG. 12).

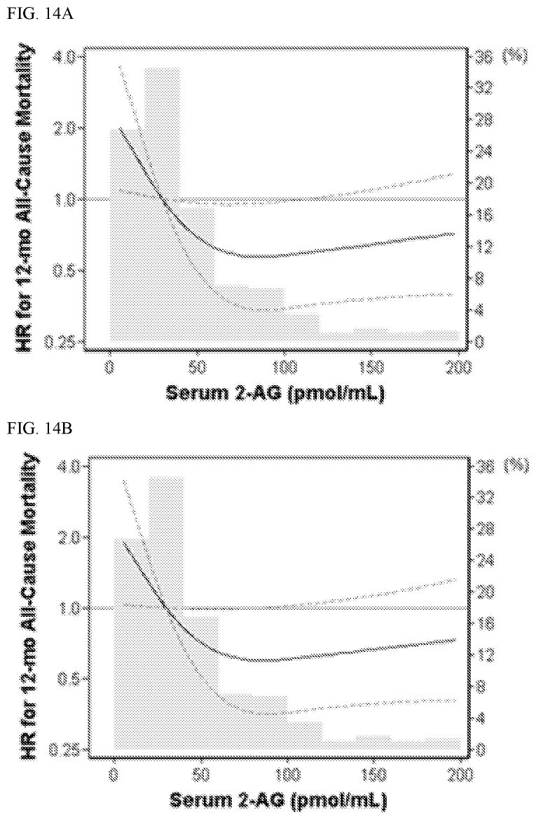

In a different set of mice, we were able to perform preliminary studies evaluating metabolic rate. The metabolic rate determinations were made using a TSE PhenoMaster System (Chesterfield, Mo.), mice were placed in metabolic cages and their CO₂ production and 02 consumption were measured to calculate their respiratory quotient and energy expenditure. We found that increasing tissue 2-AG levels were associated with a reduced rate of metabolism in preliminary studies (FIG. 13). The latter findings support our hypothesis that increasing serum 2-AG levels can reduce the risk of cachexia by decreasing the metabolic rate (n=5 in each group).

### J. REFERENCES

1. US Renal Data System: Excerpts from the USRDS. Annual Data Report: Atlas of End-Stage Renal Disease in the United States. 2012.
2. Kovesdy C P and Kalantar-Zadeh K. Enter the dragon: a Chinese epidemic of chronic kidney disease? Lancet. 2012; 379:783-5.
3. Zhang L, Wang F, Wang L, Wang W, Liu B, Liu J, Chen M, He Q, Liao Y, Yu X, Chen N, Zhang J E, Hu Z, Liu F, Hong D, Ma L, Liu H, Zhou X, Chen J, Pan L, Chen W, Wang W, Li X and Wang H. Prevalence of chronic kidney disease in China: a cross-sectional survey. Lancet. 2012; 379:815-22.
4. Chang T I, Streja E, Soohoo M, Kim T W, Rhee C M, Kovesdy C P, Kashyap M L, Vaziri N D, Kalantar-Zadeh K and Moradi H. Association of Serum Triglyceride to HDL Cholesterol Ratio with All-Cause and Cardiovascular Mortality in Incident Hemodialysis Patients. Clin J Am Soc Nephrol. 2017.
5. Doshi M, Streja E, Rhee C M, Park J, Ravel V A, Soohoo M, Moradi H, Lau W L, Mehrotra R, Kuttykrishnan S, Kovesdy C P, Kalantar-Zadeh K and Chen J L. Examining the robustness of the obesity paradox in maintenance hemodialysis patients: a marginal structural model analysis. Nephrology, dialysis, transplantation: official publication of the European Dialysis and Transplant Association—European Renal Association. 2016; 31:1310-9.
6. Obi Y, Qader H, Kovesdy C P and Kalantar-Zadeh K Latest consensus and update on protein-energy wasting in chronic kidney disease. Curr Opin Clin Nutr Metab Care. 2015; 18:254-62.
7. Ikizler T A, Cano N J, Franch H, Fouque D, Himmelfarb J, Kalantar-Zadeh K, Kuhlmann M K, Stenvinkel P, TerWee P, Teta D, Wang A Y, Wanner C, International Society of Renal N and Metabolism. Prevention and treatment of protein energy wasting in chronic kidney disease patients: a consensus statement by the International Society of Renal Nutrition and Metabolism. Kidney international. 2013; 84:1096-107.
8. Mak R H, Ikizler A T, Kovesdy C P, Raj D S, Stenvinkel P and Kalantar-Zadeh K. Wasting in chronic kidney disease. Journal of cachexia, sarcopenia and muscle. 2011; 2:9-25.
9. Thomas S S and Mitch W E. Parathyroid hormone stimulates adipose tissue browning: a pathway to muscle wasting. Curr Opin Clin Nutr Metab Care. 2017; 20:153-157.
10. Kir S, Komaba H, Garcia A P, Economopoulos K P, Liu W, Lanske B, Hodin R A and Spiegelman B M PTH/PTHrP Receptor Mediates Cachexia in Models of Kidney Failure and Cancer. Cell Metab. 2016; 23:315-23.
11. DiPatrizio N V and Piomelli D. The thrifty lipids: endocannabinoids and the neural control of energy conservation. Trends Neurosci. 2012; 35:403-11.
12. Piomelli D. The endocannabinoid system: a drug discovery perspective. Current opinion in investigational drugs. 2005; 6:672-9.
13. Maccarrone M, Bab I, Biro T, Cabral G A, Dey S K, Di Marzo V, Konje J C, Kunos G, Mechoulam R, Pacher P, Sharkey K A and Zimmer A. Endocannabinoid signaling at the periphery: 50 years after THC. Trends Pharmacol Sci. 2015; 36:277-96.
14. Silvestri C and Di Marzo V. The endocannabinoid system in energy homeostasis and the etiopathology of metabolic disorders. Cell Metab. 2013; 17:475-90.
15. Gatta-Cherifi B and Cota D. New insights on the role of the endocannabinoid system in the regulation of energy balance. Int J Obes (Loud). 2016; 40:210-9.
16. Quercioli A, Pataky Z, Vincenti G, Makoundou V, Di Marzo V, Montecucco F, Carballo S, Thomas A, Staub C, Steffens S, Seimbille Y, Golay A, Ratib O, Harsch E, Mach F and Schindler T H. Elevated endocannabinoid plasma levels are associated with coronary circulatory dysfunction in obesity. Eur Heart J. 2011; 32:1369-78.
17. Di Marzo V. The endocannabinoid system in obesity and type 2 diabetes. Diabetologia. 2008; 51:1356-67.
18. Engeli S, Bohnke J, Feldpausch M, Gorzelniak K, Janke J, Batkai S, Pacher P, Harvey-White J, Luft F C, Sharma A M and Jordan J. Activation of the peripheral endocannabinoid system in human obesity. Diabetes. 2005; 54:2838-43.
19. Despres J P, Golay A, Sjostrom L and Rimonabant in Obesity-Lipids Study G. Effects of rimonabant on metabolic risk factors in overweight patients with dyslipidemia. N Engl J Med. 2005; 353:2121-34.
20. Pi-Sunyer F X, Aronne L J, Heshmati H M, Devin J, Rosenstock J and Group RI-NAS. Effect of rimonabant, a cannabinoid-1 receptor blocker, on weight and cardiometabolic risk factors in overweight or obese patients: RIO-North America: a randomized controlled trial. JAMA. 2006; 295:761-75.
21. Nam D H, Lee M H, Kim J E, Song H K, Kang Y S, Lee J E, Kim H W, Cha J J, Hyun Y Y, Kim S H, Han S Y, Han K H, Han J Y and Cha D R. Blockade of cannabinoid receptor 1 improves insulin resistance, lipid metabolism, and diabetic nephropathy in db/db mice. Endocrinology. 2012; 153:1387-96.
22. Ebner N, Springer J, Kalantar-Zadeh K, Lainscak M, Doehner W, Anker S D and von Haehling S. Mechanism and novel therapeutic approaches to wasting in chronic disease. Maturitas. 2013; 75:199-206.
23. Friedman A N, Kim J, Kaiser S, Pedersen T L, Newman J W and Watkins B A. Association between plasma endocannabinoids and appetite in hemodialysis patients: A pilot study. Nutrition research. 2016; 36:658-62.
24. Haas M J, Mazza A D, Wong N C and Mooradian A D. Inhibition of apolipoprotein A-I gene expression by obesity-associated endocannabinoids. Obesity (Silver Spring). 2012; 20:721-9.
25. Wang A Y, Sea M M Tang N, Sanderson J E, Lui S F, Li P K and Woo J. Resting energy expenditure and subsequent mortality risk in peritoneal dialysis patients. J Am Soc Nephrol. 2004; 15:3134-43.
26. Avesani C M, Kamimura M A and Cuppari L. Energy expenditure in chronic kidney disease patients. J Ren Nutr. 2011; 21:27-30.
27. Jung K M, Clapper J R, Fu J, D'Agostino G, Guijarro A, Thongkham D, Avanesian A, Astarita G, DiPatrizio N V, Frontini A, Cinti S, Diano S and Piomelli D. 2-arachidonoylglycerol signaling in forebrain regulates systemic energy metabolism. Cell Metab. 2012; 15:299-310.
28. Tedesco L, Valerio A, Dossena M, Cardile A, Ragni M Pagano C, Pagotto U, Carruba M O, Vettor R and Nisoli E. Cannabinoid receptor stimulation impairs mitochondrial biogenesis in mouse white adipose tissue, muscle, and liver: the role of eNOS, p38 MAPK, and AMPK pathways. Diabetes. 2010; 59:2826-36.
29. Vaziri N D and Moradi H. Mechanisms of dyslipidemia of chronic renal failure. Hemodial Int. 2006; 10:1-7.
30. Korczynska J, Stelmanska E, Nogalska A, Szolkiewicz M, Goyke E, Swierczynski J and Rutkowski B. Upregulation of lipogenic enzymes genes expression in white adipose tissue of rats with chronic renal failure is associated with higher level of sterol regulatory element binding protein-1. Metabolism. 2004; 53:1060-5.
31. Chapagain A, Caton P W, Kieswich J, Andrikopoulos P, Nayuni N, Long J H, Harwood S M, Webster S P, Raftery M J, Thiemermann C, Walker B R, Seckl J R, Corder R and Yaqoob M M. Elevated hepatic 11beta-hydroxysteroid dehydrogenase type 1 induces insulin resistance in uremia. Proc Natl Acad Sci USA. 2014; 111:3817-22.
32. Osei-Hyiaman D, DePetrillo M, Pacher P, Liu J, Radaeva S, Batkai S, Harvey-White J, Mackie K, Offertaler L, Wang L and Kunos G. Endocannabinoid activation at hepatic CB1 receptors stimulates fatty acid synthesis and contributes to diet-induced obesity. J Clin Invest. 2005; 115:1298-305.
33. Gatta-Cherifi B, Matias I, Vallee M Tabarin A, Marsicano G, Piazza P V and Cota D. Simultaneous postprandial deregulation of the orexigenic endocannabinoid anandamide and the anorexigenic peptide YY in obesity. Int J Obes (Lond). 2012; 36:880-5.
34. Tetta C, David S, Biancone L, Canino F, Cambi V and Camussi G. Role of platelet activating factor in hemodialysis. Kidney Int Suppl. 1993; 39:S154-7.
35. Berdyshev E V, Schmid P C, Krebsbach R J and Schmid H H. Activation of PAF receptors results in enhanced synthesis of 2-arachidonoylglycerol (2-AG) in immune cells. FASEB J. 2001; 15:2171-8.
36. Dotsey E Y, Jung K M, Basit A, Wei D, Daglian J, Vacondio F, Armirotti A, Mor M and Piomelli D. Peroxide-Dependent MGL Sulfenylation Regulates 2-AG-Mediated Endocannabinoid Signaling in Brain Neurons. Chem Biol. 2015; 22:619-28.
37. Igarashi M, DiPatrizio N V, Narayanaswami V and Piomelli D. Feeding-induced oleoylethanolamide mobilization is disrupted in the gut of diet-induced obese rodents. Biochim Biophys Acta. 2015; 1851:1218-26.
38. Rhee C M, Nguyen D V, Moradi H, Brunelli S M, Dukkipati R, Jing J, Nakata T, Kovesdy CP, Brent G A and Kalantar-Zadeh K Association of Adiponectin With Body Composition and Mortality in Hemodialysis Patients. American journal of kidney diseases: the official journal of the National Kidney Foundation. 2015; 66:313-21.
39. Feroze U, Noori N, Kovesdy C P, Molnar M Z, Martin D J, Reina-Patton A, Benner D, Bross R, Norris K C, Kopple J D and Kalantar-Zadeh K Quality-of-life and mortality in hemodialysis patients: roles of race and nutritional status. Clinical journal of the American Society of Nephrology: CJASN. 2011; 6:1100-11.
40. Moradi H, Oveisi F, Khanifar E, Moreno-Sanz G, Vaziri N D and Piomelli D. Increase Renal 2-Arachidonoylglycerol level is Associate with Improved Renal Function in a Mouse Model of Acute Kidney Injury. Cannabis and Cannabinoid Research. 2016; 1; 218-228.
41. Scalvini L, Piomelli D and Mor M Monoglyceride lipase: Structure and inhibitors. Chemistry and Physics of Lipids. 2015:197; 13-24.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating chronic kidney disease in a subject in need thereof, the method comprising administering an effective amount of an agent that increases the level of activity of a cannabinoid receptor to the subject.

2. The method of claim 1, wherein the cannabinoid receptor is human cannabinoid receptor type 1.

3. The method of claim 1, wherein the agent is an agonist of a cannabinoid receptor.

4. The method of claim 1, wherein the agent is an agonist of human cannabinoid receptor type 1.

5. The method of claim 1, wherein the agent is an endocannabinoid.

6. The method of claim 3, wherein the agonist is tetrahydrocannabinol or a derivative thereof, 2-arachidonoyl-sn-glycerol (2-AG) or a derivative thereof, cannabidiol or a derivative thereof, or cannabis extract.

7. The method of claim 3, wherein the agonist is 2-arachidonoyl-sn-glycerol (2-AG).

8. The method of claim 1, wherein the agent inhibits the degradation of an agonist of a cannabinoid receptor.

9. The method of claim 8, wherein the agent is an inhibitor of monoacylglycerol lipase (MGL).

10. The method of claim 8, wherein the agent is URB602, N-arachidonoyl maleimide, JZL184 (4-nitrophenyl-4-(dibenzo[d][1,3]dioxol-5-yl(hydroxy)methyl)piperidine-1-carboxylate), JZL195, KML29, SAR127303, JJKK-048, MJN110, CL6a, Comp21, N-octylbenzisothiazolinone, octhilinone, NAM, dicyclopentamethylenethiuram disulfide, pristimerin, or euphol.

11. The method of claim 8, wherein the agent is URB602, N-arachidonoyl maleimide, JZL184 (4-nitrophenyl-4-(dibenzo[d][1,3]dioxol-5-yl(hydroxy)methyl)piperidine-1-carboxylate), JZL195, KML29, SAR127303, JJKK-048, MJN110, CL6a, or Comp21.

12. The method of claim 8, wherein the agent is N-octylbenzisothiazolinone, octhilinone, NAM, dicyclopentamethylenethiuram disulfide, pristimerin, or euphol.

13. The method of claim 8, wherein the agent is URB602, N-arachidonoyl maleimide, or JZL184 (4-nitrophenyl-4-(dibenzo[d][1,3]dioxol-5-yl(hydroxy)methyl)piperidine-1-carboxylate).

14. A method of treating chronic kidney disease in a subject in need thereof, the method comprising administering an effective amount of an agent that increases the serum level of 2-arachidonoyl-sn-glycerol (2-AG), to the subject.

15. The method of claim 14, wherein the agent is 2-arachidonoyl-sn-glycerol (2-AG).

16. The method of claim 14, wherein the agent reduces the degradation of 2-arachidonoyl-sn-glycerol (2-AG).

17. The method of claim 16, wherein the agent is an inhibitor of monoacylglycerol lipase (MGL).

18. The method of claim 17, wherein the agent is URB602, N-arachidonoyl maleimide, JZL184 (4-nitrophenyl-4-(dibenzo[d][1,3]dioxol-5-yl(hydroxy)methyl)piperidine-1-carboxylate), JZL195, KML29, SAR127303, JJKK-048, MJN110, CL6a, Comp21, N-octylbenzisothiazolinone, octhilinone, NAM, dicyclopentamethylenethiuram disulfide, pristimerin, or euphol.

19. The method of claim 17, wherein the agent is URB602, N-arachidonoyl maleimide, or JZL184 (4-nitrophenyl-4-(dibenzo[d][1,3]dioxol-5-yl(hydroxy)methyl)piperidine-1-carboxylate).

20. The method of claim 14, wherein the agent is a precursor in the biosynthesis of 2-arachidonoyl-sn-glycerol (2-AG).

21. The method of claim 20, wherein the agent is 1-palmitoyl-2-arachidonoyl-sn-glycerol.

22. The method of claim 14, wherein the serum level of 2-arachidonoyl-sn-glycerol (2-AG) is increased in the subject is increased to greater than 117.16 pmol/mL.

23. The method of claim 1, wherein the chronic kidney disease is end stage renal disease.

24. The method of claim 1, wherein the subject has cachexia.

25. A method of identifying the subject of one of claims 1 to 24, comprising detecting the serum level of 2-arachidonoyl-sn-glycerol (2-AG) in a candidate subject; wherein the candidate subject is identified as a subject by detection of a serum level of 2-arachidonoyl-sn-glycerol (2-AG) less than 55.97 pmol/mL in the subject.
